(19) United States
(12) Patent Application Publication
(10) Pub. No.: US 2017/0128486 A1
(43) Pub. Date: May 11, 2017

(54) CONSUMABLE COMPOSITIONS AND USES THEREOF FOR ALLEVIATING UNDESIRABLE PHYSIOLOGICAL EFFECTS SYSTEMS

(71) Applicant: Julie Desbarats, Montreal (CA)

(72) Inventor: Julie Desbarats, Montreal (CA)

(73) Assignee: ImmunoL0G1C R&D Inc., Montreal (CA)

(21) Appl. No.: 14/938,475

(22) Filed: Nov. 11, 2015

(51) Int. Cl. A61K 33/02; A61K 33/00; C01C 1/16; A61K 33/20

(52) U.S. Cl. CPC A61K 33/02; A61K 33/20; A61K 33/00; C01C 1/164

(57) ABSTRACT

A functional chemical consumption formulated for human consumption and useful for alleviating undesirable physiological effects. The chemical composition may be in liquid, food, or pill form and includes ingredients specifically to provide comfort for persons exposed to higher altitudes. Primary ingredients may include a combination of ammonium, chloride, and potassium ion.

### CROSS-REFERENCE TO RELATED APPLICATION

The present application is related to and claims priority from prior provisional application Ser. No. 62/078,481, filed Nov. 12, 2014 which application is incorporated herein by reference.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of health consumables and more specifically relates to a functional health beverage or food for alleviating undesirable physiological effects.

2. Description of Related Art

Airline travel exposes passengers to many physiological stressors, including low cabin air pressure, high ambient carbon dioxide levels, extremely low humidity, undesirably high concentrations and variety of microorganisms (due to the confined space, the low volume of air per person and the recycled cabin air), prolonged inactivity, and disruption of circadian rhythms. These stressors lead to adverse effects including hypoxia, decreased tissue oxygenation, respiratory alkalosis, dehydration and immune suppression. Therefore, a significant population of passengers acquires viral infections during airline travel and virtually all passengers experience dehydration and fatigue as result of the adverse physiological changes induced by airline travel.

People who are subjected to low ambient air pressure and consequently to a low partial pressure of oxygen, including airline passengers and those recently arrived at high altitude, experience hypoxia and decreased tissue oxygenation. These people automatically compensate for hypoxia by increasing their respiratory rate. This increased respiratory rate leads to respiratory alkalosis (i.e. an increase in the pH of body fluids due to “blowing off” of additional carbon dioxide). Respiratory alkalosis in turn leads to a cycle of autonomically decreased ventilation, worsening hypoxia, and reduced tissue oxygenation: specifically, decreased amounts of oxygen are dissolved in the blood (decreased partial pressure of arterial oxygen, PaO₂) leading to decreased hemoglobin oxygen saturation (decreased O₂ sats). Hypoxia occurs even in healthy people when they ascend to high altitude, and in severe cases this can result in Acute Mountain Sickness. Even mild hypoxia can cause symptoms including headache, fatigue, light-headedness, numbness or tingling of the extremities, and nausea. Alkalosis further leads to downstream metabolic disturbances including drops in serum potassium and calcium, which in turn can lead to muscle cramping and discomfort, especially with the imposed immobility of airline travel. Symptoms of severe alkalosis can include confusion, stupor, tremor, lightheadedness, muscle twitching, nausea, and numbness or tingling in the face, hands, or feet. Even at pre-clinical levels, mild alkalosis causes fatigue and headache, and hampers immune function.

Dehydration results from an excessive loss of water from body tissues. During airline travel, dehydration is caused by the extreme dryness of recycled cabin air and the increased respiratory rate imposed by low cabin pressure. Dehydration may be exacerbated by passengers reducing their consumption of fluids due to the constraints of airline travel. Symptoms of dehydration include headache, dry mouth, dry eyes, itchy dry skin and, in severe cases, hypotension and/or syncope. Furthermore, dehydration contributes to fatigue, exacerbates alkalosis (via contraction alkalosis) and compromises immunity.

Immune-mediated protection from disease is further challenged in airline passengers due to high passenger density, small volume of air per passenger, recycled air, inevitable contact with shared surfaces and fomites and unaccustomed diversity of microorganisms, particularly during international travel. Furthermore, immune function is suppressed by alkalosis and dehydration, and by physiological and emotional stress, which is commonly experienced during today's airline travel. It has been documented that contagion rates among airline passengers surpass those within families, among shipboard passengers, and in commuters regularly using rush hour public transportation such as subways.

Traditionally airlines have offered a limited range of beverages to passengers, including water, soft drinks, fruit juices, tea or coffee, and occasionally alcoholic beverages. Because plain water is lacking solutes (e.g. sugars and electrolytes) it provides poor rehydration and it does not correct hypoxia nor alkalosis. Non-caffeinated soft drinks can provide good rehydration but if anything, their carbonation contributes to the CO₂ overload experienced by airline passengers, without sufficiently addressing passengers' respiratory alkalosis and hypoxia. Dilute fruit juices may be superior to water as a rehydration beverage in an exercising subject. However, unlike a perspiring athlete, airline passengers become dehydrated through loss of water from insensible perspiration and respiration, with minimal loss of electrolytes. Therefore the hypertonic nature of fruit juice in which solutes are far more concentrated than in blood is not well suited to provide optimal rehydration to immobilized passengers. Furthermore, fruit juices are digested and metabolized without causing a significant change in body fluid pH and therefore do not specifically counter the alkalosis and hypoxia of airline passengers. Caffeinated beverages worsen dehydration due to their diuretic properties and they can also further disrupt sleep patterns and increase anxiety and restlessness. Alcoholic beverages are dehydrating as well because water molecules are utilized during alcohol metabolism and detoxification in the liver. Neither alcoholic nor caffeinated beverages help to counter alkalosis and hypoxia, or immune suppression.

Recently certain airlines have recognized the damaging effects of air travel on passengers' health and these airlines have started serving functional foods and drinks in-flight, to remediate these damaging effects. Examples of such drinks include Flyhidrate™ (www.flyhidrate.com), FlyFit™ from Vitalit Laboratories (www.vitalithealth.com), and 1Above™ (www.flylabove.com). While these drinks are targeted at improving blood circulation and at fighting free radical damage from cosmic radiation, none of these known beverages addresses the key issues of pH imbalance and hypoxia that are caused by airline travel and/or high altitude.

Additional types of compositions for addressing problems of dehydration and/or alkalosis include those described in Indian patent no. 247946, U.S. Pat. No. 4,729,894 and international patent publication WO 2013/093863. However, none of these compositions provides the required constituents for addressing alkalosis, hypoxia and dehydration, let alone addressing alkalosis, hypoxia and dehydration caused by airline travel and/or high altitude.

Accordingly, there is a need for a solution to the discomfort, fatigue, and eventual illnesses experienced by airline passengers and people exposed to low partial pressure of oxygen (e.g. high altitude over 1,500 meters and more above sea level).

There is also a need for effective remedial strategies that target upstream factors responsible for initiating adverse effects on the health of airline passengers.

There is particularly a need for oral compositions able to correct and/or to prevent respiratory alkalosis, hypoxia, dehydration and/or immune suppression.

Several attempts have been made to solve the above-mentioned problems such as those found in U.S. Pat. No. 4,729,894 to Teeter; U.S. Pat. No. 6,682,762 to Register; and U.S. Pat. No. 7,827,015 to McGoogan. This art is representative of animal feed additives seeking to correct alkalosis. However, none of the above inventions and patents, taken either singly or in combination, is seen to describe the invention as claimed.

Preferably, a functional health beverage for alleviating undesirable physiological effects should provide a consumable health beverage incorporating compounds that function in combination to alleviate undesirable physiological effects and, yet would operate reliably and be manufactured at a modest expense. Thus, a need exists for a reliable functional health beverage for alleviating undesirable physiological effects to avoid the above-mentioned problems.

### BRIEF SUMMARY OF THE INVENTION

In view of the foregoing disadvantages inherent in the known health beverage art, the present invention provides a novel functional health beverage for alleviating undesirable physiological effects. The general purpose of the present invention, which will be described subsequently in greater detail, is to provide comfort for those ingesting the product. The present composition may also be added to any potable liquid or edible food or incorporated into a tablet or capsule, to be consumed preferably with a suitable amount of water or other suitable liquid.

The invention concerns an edible or potable composition, comprising a combination of (i) ammonium, (ii) chloride, and (iii) potassium. The composition may further comprise sodium. The composition may also further comprise glucose and/or galactose. The composition may further comprise magnesium, calcium and/or zinc. The composition may further comprise at least one other ingredient to promote immune function. The composition may further comprise L-glutamine or 5-hydroxytryptophan. Preferably the composition is formulated for human consumption and it is used for alleviating undesirable physiological effects caused by exposures to low air pressure for example, airline travel and/or high altitude.

According to one embodiment, the composition is in a form of an aqueous solution, in which the ions are soluble and are derived from solubilisation of water-soluble salts. In one embodiment, the aqueous solution comprises about 0.006% w/v to about 0.07% w/v of ammonium ion, about 0.04% w/v to about 0.9% w/v of chloride ion and about 0.01% w/v to about 0.15% w/v of potassium ion. In one embodiment, the aqueous solution comprises about 0.01% w/v to about 0.3% w/v of sodium ion. In one embodiment, the aqueous solution comprises about 0.1% w/v to about 6% w/v of glucose and/or galactose.

The invention also concerns a potable liquid beverage. Preferably the beverage is formulated for human consumption and it is used for alleviating undesirable physiological effects caused by airline travel and/or exposure to high altitude.

The invention also concerns containers, comprising suitable water-soluble ingredients in desirable amounts for alleviating undesirable physiological effects caused by airline travel and/or exposure to high altitude. In one embodiment, the container comprises ammonium chloride and potassium chloride as a source of ammonium, chloride and potassium. In embodiments, the container further comprises sodium chloride, magnesium chloride, calcium chloride, and/or zinc chloride. The container may be a package, a pouch, a spout pouch, a carton, a sachet, a packet, a can or a bottle.

The composition, beverage and/or container according to the invention may further comprise flavoring agents, coloring agents, sweeteners, stabilizers, preservatives, acidifying agents, excipients and the like.

The invention also concerns kits and methods of uses, including a method for alleviating undesirable physiological effects caused by airline travel and/or exposure to high altitude.

Yet, the invention further concerns processes for making compositions and beverages as defined herein.

An advantage of the oral compositions, beverages, containers, packages, methods and kits according to the present invention is that they target elevation of serum pH, hypoxia, and dehydration, three upstream factors that initiate the adverse effects on the health of airline passengers and people having to adapt to high altitude.

Another advantage of the invention relates to the fact that it further provides effective, convenient and inexpensive solutions to discomfort, fatigue and/or illnesses experienced by airline passengers and/or people exposed to low partial pressure of oxygen (e.g. high altitude over 1,500 meters and more above sea level).

An embodiment of the present invention may comprise a consumable composition comprising ammonium formulated for human consumption where said consumable composition is useful for alleviating undesirable physiological effects caused by exposure to low air pressure which occurs during air travel and alternatively at altitudes above about 1,500 meters (about 5,000 feet) above sea level. The consumable composition is useful for alleviating said undesirable physiological effects comprising alkalosis and hypoxia. The consumable composition further comprises chloride and potassium. A combination of said ammonium, said chloride, and said potassium is useful for alleviating alkalosis and alternatively hypoxia.

An embodiment of the present invention may further comprise a container for retaining a consumable composition, which may comprise a container body and an inner volume; wherein said container body and said inner volume structurally comprise said container for retaining said consumable composition; wherein said consumable composition comprises ammonium chloride and potassium chloride; wherein said ammonium chloride and said potassium chloride function in chemical combination for alleviating undesirable physiological effects; and wherein said container body proximate to said ammonium chloride and said potassium chloride is useful for preserving an efficacy of said consumable composition.

The present invention holds significant improvements and serves as a functional health beverage or composition for alleviating undesirable physiological effects. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, functional health beverage and/or food for alleviating undesirable physiological effects, constructed and operative according to the teachings of the present invention.

FIG. 1 shows a functional health beverage for alleviating undesirable physiological effects being consumed by a female passenger on an airplane according to an embodiment of the present invention.

FIG. 2 shows a chart illustrating a comparison of hemoglobin oxygen saturation of the same subject during two separate commercial flights: Flight 1 (dotted line, hollow circles) during which placebo beverage was consumed, and Flight 2 (solid line, black squares) during which Test Beverage according to invention was consumed.

FIG. 3 is a graph which shows the average and standard error of the minimum measured oxygen saturation during flight by three subjects drinking control beverages (hashed bar) or Test Beverage according to invention (solid bar).

FIG. 4 illustrates in chart form the kinetics of oxygen saturation as was monitored during a long-haul flight (six hours, 20 minutes) during which 2.5 servings of beverage according to invention were consumed, one-half serving (0.5 dose, arrows) at a time.

FIG. 5 shows a chart which illustrates one subject's self-reported comfort and wellbeing scores for six parameters (general wellbeing, absence of headache, hydration, muscle comfort, energy, and alertness) during the course of two flights, Flight 1 with complimentary airline beverages (hashed bars) and Flight 2 with Test beverage according to invention (solid black bars).

FIG. 6 shows a chart depicting the aggregate comfort index for three separate subjects (a), and the average for the 3 subjects (b), on flights with complimentary airline beverages (hashed bars) versus Test beverages according to invention (solid black bars).

The various embodiments of the present invention will hereinafter be described in conjunction with the appended drawings, wherein like designations denote like elements.

### DETAILED DESCRIPTION

As discussed above, embodiments of the present invention relate to a health beverage and more particularly to a functional health beverage for alleviating undesirable physiological effects as used to improve comfort during airline travel and exposure to altitude.

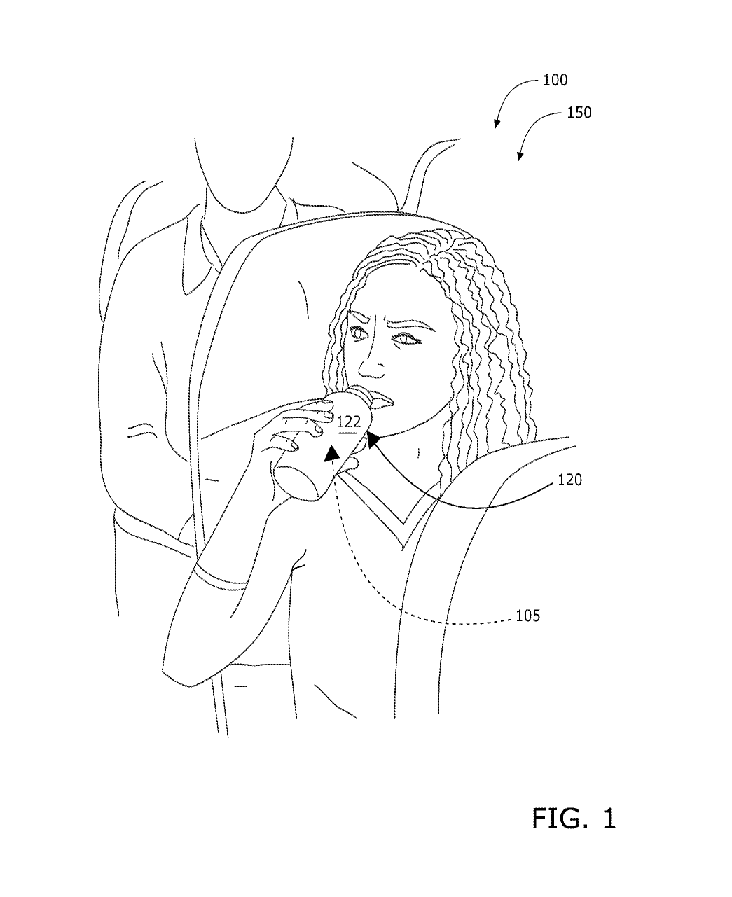

Generally speaking in reference to the drawings there is shown in the FIGS. 1-6 consumable compositions and uses thereof for alleviating undesirable physiological effects systems 100 according to the present invention.

Beginning with FIG. 1, there is shown an embodiment of consumable compositions and uses thereof for alleviating undesirable physiological effects systems 100 during ‘in-use’ condition 150. As shown, passenger 140 traveling on an airplane is drinking consumable composition 105 from container 120.

In an embodiment of the present invention, consumable composition 110 may comprise ammonium. Further, consumable composition 110 comprising ammonium may be formulated for human consumption, as shown, in FIG. 1. As may be appreciated, consumable composition 110 comprising ammonium may be useful for alleviating undesirable physiological effects caused by exposure to low air pressure which occurs during air travel and alternatively at altitudes above about 5,000 feet. More specifically, consumable composition 110 comprising ammonium may be useful for alleviating undesirable physiological effects such as alkalosis and hypoxia.

In other embodiments of the present invention, consumable composition 110 may comprise chloride and potassium. As may be appreciated, consumable composition 110 comprising a combination of ammonium, chloride, and potassium may be useful for alleviating alkalosis and alternatively hypoxia.

In referring generally to FIGS. 1-6, consumable composition 110 may comprise, in yet another embodiment, at least about 0.006% w/v ammonium, at least about 0.04% w/v chloride, and at least about 0.15% w/v potassium. At least about 0.006% w/v ammonium, at least about 0.04% w/v chloride, and at least about 0.15% w/v potassium comprises in chemical combination consumable composition 110. Further, consumable composition 110 is formulated for human consumption and useful for alleviating undesirable physiological effects. In another embodiment, consumable composition 110 may further comprise sodium. In yet another embodiment, consumable composition 110 may further comprise at least one sweetener and alternatively at least one sugar. In yet another embodiment, consumable composition 110 may further comprise at least one flavoring agent useful for adding flavor to consumable composition 110. In yet another embodiment, consumable composition 110 may further comprise at least one coloring agent useful for adding color to consumable composition 110. In yet another embodiment, consumable composition 110 may further comprise at least one preservative useful for preserving consumable composition 110 over a period of time.

In one embodiment of the present invention, consumable compositions and uses thereof for alleviating undesirable physiological effects systems 100 may comprise container 120 for retaining consumable composition 110. Container 120 may comprise container body 122 and an inner volume. Container body 122 and the inner volume may structurally comprise container 120 for retaining consumable composition 110. In one embodiment, consumable composition 110 comprises ammonium chloride and potassium chloride. As noted, ammonium chloride and potassium chloride may function in chemical combination for alleviating undesirable physiological effects. As may be appreciated, container body 122 proximate to ammonium chloride and potassium chloride may be useful for preserving an efficacy of consumable composition 110. Naturally, consumable composition 110 is useful for human consumption.

In other embodiments of the present invention, container body 122 of container 120 may further comprise sodium useful for preserving efficacy of consumable composition 110. In other embodiments, container body 122 may further comprise magnesium useful for preserving efficacy of consumable composition 110. In yet other embodiments, container body 122 may further comprise calcium useful for preserving efficacy of consumable composition 110. In yet other embodiments, container body 122 may further comprise zinc useful for preserving efficacy of consumable composition 110.

As may further be noted and appreciated, in some embodiments, container 120 may comprise a package and alternatively a pouch useful for retaining consumable composition 110 comprising a non-aqueous solution. In other embodiments, container 120 may comprise a can and alternatively a bottle useful for retaining consumable composition 110 comprising an aqueous solution (as shown in FIG. 1).

In an embodiment of consumable compositions and uses thereof for alleviating undesirable physiological effects systems 100, the invention may comprise a kit. The kit may comprise container 120, consumable composition 110, and a set of user instructions.

In another embodiment of consumable compositions and uses thereof for alleviating undesirable physiological effects systems 100, the kit may comprise at least two container bodies 122, each container body(s) 122 comprising consumable composition 110, and a set of user instructions.

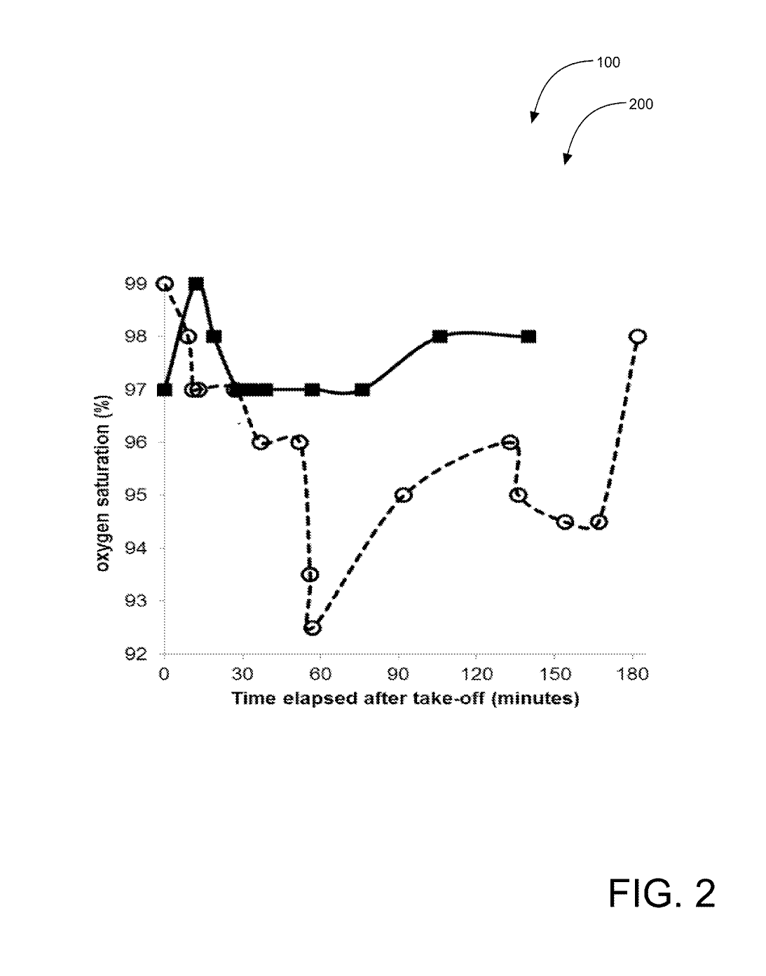

Referring now to FIG. 2 showing chart 200 illustrating a comparison of hemoglobin oxygen saturation of the same subject during two separate commercial flights: flight 1 (dotted line, hollow circles) during which placebo beverages were consumed; and flight 2 (solid line, black squares) during which Test Beverages according to invention were consumed.

As shown, during each flight, the subject drank two 500 ml doses of beverage, either placebo (dotted line, hollow circles) or Test Beverage (solid line, black squares). In both cases, the first dose was consumed within 10 minutes of take-off (time=0), and the second dose was consumed slowly beginning one hour after take-off (time=60). During the placebo flight (dotted line, hollow circles), note the rapid drop-off followed by up-and-down cycling of oxygen saturation. In contrast, the Test Beverage (solid line, black squares) allowed the subject to maintain a high and stable level of oxygen saturation, at or above 97%. The Test beverage flight departed a high-elevation city (airport at around 7,000 feet or 2,200 meters), which accounts for the starting oxygen saturation of 97% for that flight; when the first dose of Test Beverage was rapidly consumed during the first 10 minutes of flight, the oxygen saturation rose quickly to 99%, despite the dropping air pressure as the aircraft gained altitude. For both flights, the final measurement was taken upon landing near sea level, therefore normal oxygen saturation of 98% was re-established at that point, independent of beverage consumed. The present figure is in relation to an embodiment of the present invention.

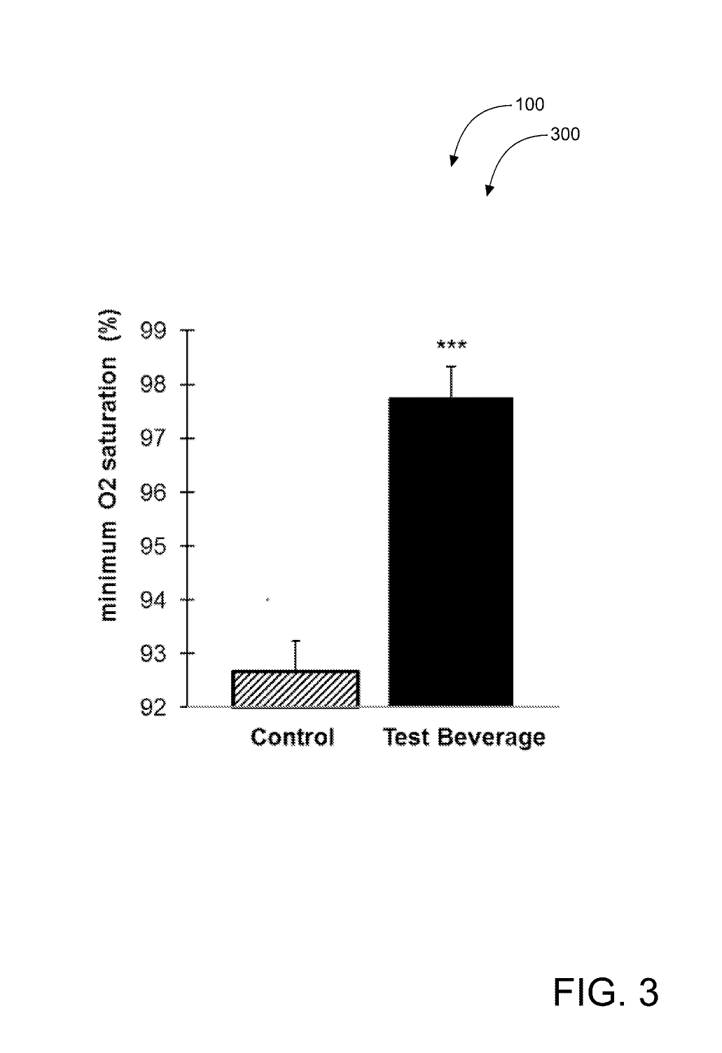

Referring now to FIG. 3 showing graph 300 which shows the average and standard error of the minimum measured oxygen saturation during flight by three subjects drinking control beverages (hashed bar) or Test Beverage according to invention (solid bar). Subjects demonstrated significantly (***, p<0.0001) higher oxygen saturations during flights when they drank Test Beverages, compared to flights when they consumed a variety of control beverages Minimum measured oxygen saturation levels of 92-94% in subjects drinking control beverages are consistent with published values in the literature for passengers on commercial flights. The Test Beverage prevented the drop in oxygen saturation and allowed passengers to maintain normal, healthy oxygen saturations of 97% and above.

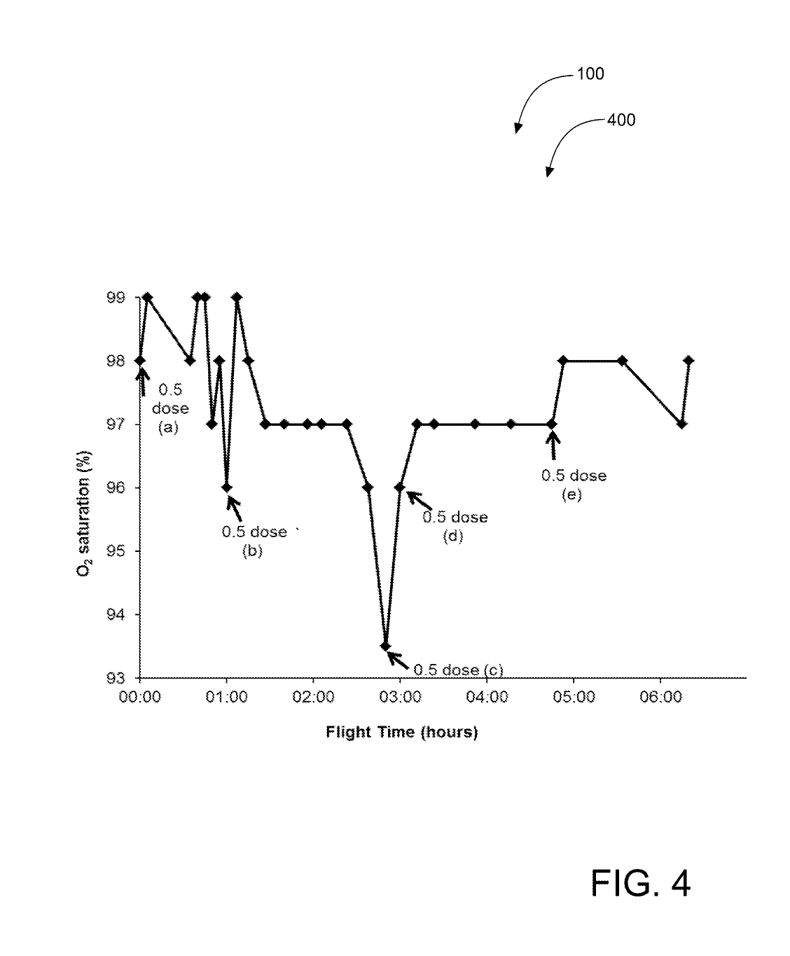

Referring now to FIG. 4 illustrating chart 400 showing kinetics of oxygen saturation as monitored during a long-haul flight (six hours, 20 minutes) during which 2.5 servings of beverage according to invention were consumed, one-half serving (0.5 dose, arrows) at a time. The first half-dose (a) was consumed immediately before take-off at time 0. The next portion (b) was withheld until oxygen saturation began to drop, in order to determine the effective duration of the half-serving of Test beverage. Oxygen saturation rose in response to the test beverage, and no further Test beverage was consumed until oxygen saturation once again dropped. This defined the effective duration of a full dose of beverage (ie. the combination of 2 half doses (a) and (b)). At this point a full dose of beverage was administered in 2 half doses (c) and (d) allowing only a short interval in between to measure oxygen saturation and allow the subject time to ingest the drink comfortably. Again, the oxygen saturation recovered rapidly after the Test beverage was consumed. The final half dose (e) of Test beverage was administered when a steady oxygen saturation of 97% had been maintained for 1.5 hours without additional Test Beverage, and before the aircraft began to descend, to determine whether a further increase in oxygen saturation above 97% could be obtained. The final measurement was recorded once the aircraft had landed.

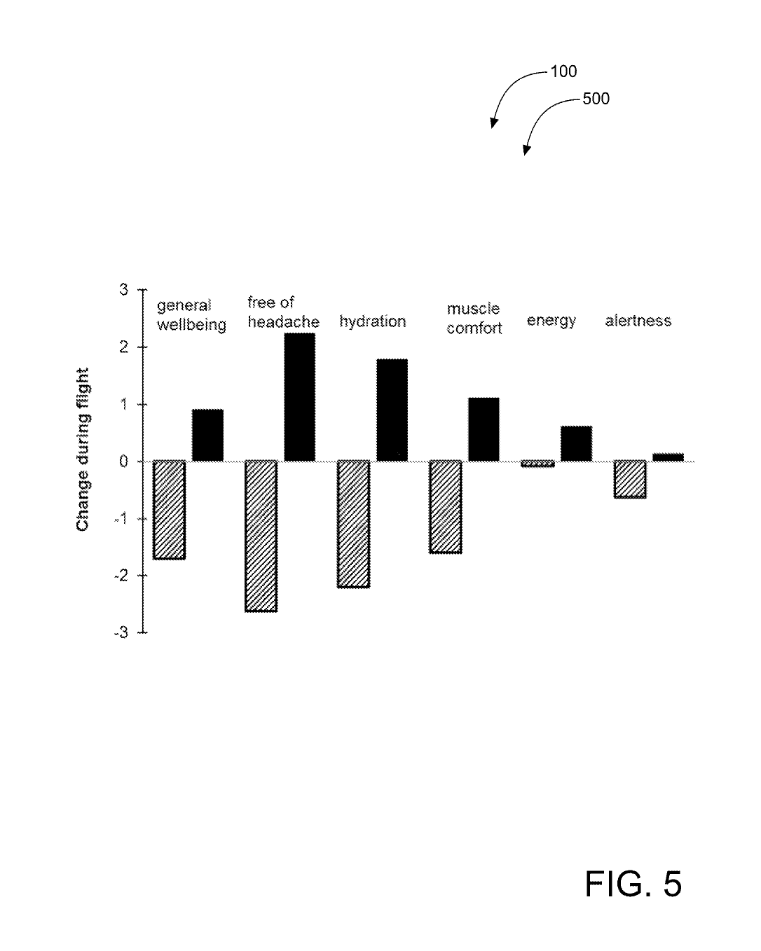

Referring now to FIG. 5 showing a chart 500 which illustrates one subject's self-reported comfort and wellbeing scores for six parameters (general wellbeing, absence of headache, hydration, muscle comfort, energy, and alertness), during the course of two flights. A flight with complimentary airline beverages (hashed bars) was compared with the return flight with Test beverage according to invention (solid black bars). Negative scores represent a decrease in comfort from the beginning to the end of the flight. Positive scores indicate that comfort has increased during the flight. Neutral (near zero) scores indicate that comfort remained unchanged during the fight. The chart illustrates a reversal from increasing discomfort during the flight with airline beverages (hashed bars), to increasing comfort over the course of a flight during which the Test Beverage was consumed (solid black bars).

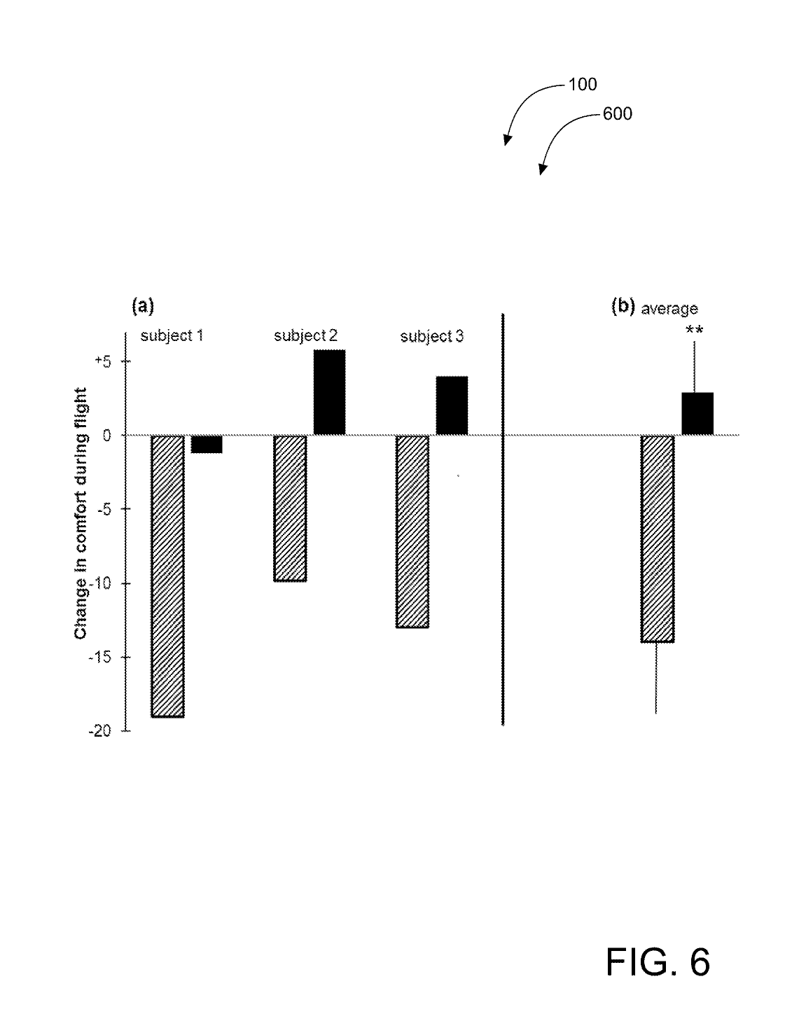

Referring now to FIG. 6 showing a chart 600 of the aggregate comfort index for three separate subjects (a), and the average and standard error for the 3 subjects (b), on flights with complimentary airline beverages (hashed bars) versus Test beverages according to invention (solid black bars). Each subject rated six comfort parameters (general wellbeing, absence of headache, hydration, muscle comfort, energy, and alertness) at the beginning and end of each of the two flights. The change in comfort over the course of each flight was determined for each parameter and a global score representing the sum of all parameter scores was calculated for each passenger (see below for details). A negative score represents a decrease in global comfort and wellbeing during the course of the flight, as occurred for all passengers on flights with airline beverages. A positive score represents an increase in comfort over the course of the flight. Error bars show the standard error of the mean score for the 3 subjects. There is a significant increase in comfort scores between flights with airline beverages versus Test beverages (**, p=0.0015).

Referring now to A) Compositions of the invention and uses thereof: The invention concerns edible or potable compositions (e.g. as a liquid beverage, or as a powder, paste, gel, concentrate, tablet, effervescent tablet, capsule, or the like to be diluted into liquid, incorporated into food, and/or swallowed with a suitable amount of water or other liquid) and methods of using same, for alleviating undesirable physiological effects caused by airline travel and/or exposure to high altitude (e.g. over about 1,500 meters above sea level or above 2,400 meters above sea level). According to more specific aspects, the invention aims to treat elevation of serum pH (alkalosis) and alleviate hypoxia (decreased hemoglobin oxygen saturation and/or partial pressure of oxygen dissolved in serum) and to treat dehydration caused by airline travel and high altitude in subjects in need thereof.

As used herein, the “subject” includes living organisms in which respiratory alkalosis can occur, or which are susceptible to such condition, particularly when exposed to low ambient air pressure and consequently to a low partial pressure of oxygen. The term “subject” includes animals such as mammals or birds. Preferably, the subject is a mammal, including but not limited to human, domestic animals (e.g. bovine, porcine, equine, canine, feline), laboratory animals (e.g. rat, mouse, rabbit, etc.) and wild animals such as those living in zoos (e.g. lion, tiger, elephant, and the like). More preferably, the subject is a human. In one embodiment, the subject is an airline passenger or crew. In another embodiment, the subject is a person recently arrived at high altitude (e.g. an altitude over about 1,500 meters above sea level, preferably over 2,400 meters above sea level), including but not limited to visitors adapting to high altitude and people engaging in an athletic activity at high altitude (e.g. climbing).

As used herein, the terms “edible” and “potable” are used interchangeably, depending if the composition is a solid (e.g. a powder) or an aqueous solution (e.g. a beverage), and they refer to a composition that is safe enough to be consumed by a subject (e.g. humans) or to be used with low risk of immediate or long term harm.

As used herein, the term “alkalosis” refers to a physiological condition reducing normal hydrogen ion concentration of arterial blood plasma (alkalemia). The range of blood pH considered clinically normal is 7.35 to 7.45, more preferably 7.38 to 7.42. Alkalosis occurs when the serum pH is 7.45 or higher. Alkalosis is usually divided into the categories of respiratory alkalosis and metabolic alkalosis or combinations thereof. Although it is conceivable that compositions of the present invention be useful for metabolic alkalosis, it is typically directed to treat/prevent respiratory alkalosis. In embodiments, the term “alkalosis” encompasses a pH greater or equal to 7.42, or preferably greater than 7.45. As used herein, the term “hypoxia” refers to a decreased supply of oxygen to the cells and tissues of the body, including decreased amounts of oxygen dissolved in body fluids, such as in arterial blood (partial pressure of oxygen in arterial blood, PaO₂); and/or decreased hemoglobin oxygen saturation. Normal hemoglobin oxygen saturation levels in humans are generally considered to be 95-100%, more preferably 97-99%.

As used herein, the term “dehydration” loosely refers to any condition where intracellular and/or extracellular fluid volume is reduced; including hypernatremia (loss of free water and the attendant excess concentration of salt), and hypovolemia (loss of blood volume, particularly plasma). In preferred embodiments, the invention aims to achieve or maintain a desirable state of hydration, or avoid the onset of clinical signs of dehydration, which include increased thirst, delayed central capillary refill time (>2 seconds), decreased tissue turgor, and decreased urine output.

The terms “treatment” or “treating” of a subject include the application or administration of a composition of the invention to a subject with the purpose of delaying, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” encompasses “prevention” and it refers to any indication of success in the treatment or amelioration of an injury, pathology or condition, including any objective or subjective parameter such as abatement; remission; lessening of the rate of worsening; lessening severity of the condition; stabilization, diminishing of symptoms or making the injury, pathology or condition more tolerable to the subject; slowing in the rate of degeneration or decline; making the final point of degeneration less debilitating; or improving a subject's physical or mental well-being. In some embodiments, the term “treating” can include increasing a subject's life expectancy and/or delay before additional treatments are required. In preferred embodiments, “treatment” or “treating”, in association with “alkalosis” refers to achieving or maintaining a plasma pH in the range considered physiologically normal, that is, between 7.35 and 7.45, or more preferably, between 7.38 and 7.42. In preferred embodiments, “treatment” or “treating”, in association with “hypoxia” refers to increasing the amount of oxygen dissolved in plasma, and/or increasing the haemoglobin oxygen saturation; where possible given the external air pressure and health of the subject, achieving or maintaining a hemoglobin oxygen saturation considered physiologically normal, that is, above 94%, or more preferably, 97% or greater.

The term “high altitude” is sometimes defined to begin at 2,400 meters (8,000 feet) above sea level. At high altitude, atmospheric pressure is lower than that at sea level and most people begin to develop noticeable discomfort related to high altitude/low air pressure between about 2,400 meters (about 8,000 feet) and about 3,000 meters (about 10,000 feet) above sea level or at an equivalent air pressure in flight, although measurable physiological changes and some discomfort may appear at lower elevation, eg 1,500 meters (about 5,000 feet) or even lower, depending on the health, activity level, and sensitivity of the individual. Between about 4,000-4,500 meters (about 14,000-15,000 feet) elevation, over 50% of moderately active adults develop one or more symptoms of clinical Acute Mountain Sickness. By way of example, there are 53 peaks between 14,000 and 15,000 feet elevation in Colorado alone, and the peak of Mount Everest is 8,848 meters (29,029 feet) above sea level. In embodiments, the term “high altitude” refers to an altitude of about 1,500 meters or more above sea level, or about 2,000 meters or more above sea level, or about 2,400 meters or more above sea level, or about 3,000 meters or more above sea level, or about 5,000 meters or more above sea level.

One of the main aspects of the invention is directed to edible or potable compositions for treating or preventing alkalosis, particularly respiratory alkalosis caused by airline travel and high altitude. Accordingly, one aspect of the invention concerns an edible or potable composition comprising a combination of (i) ammonium ion, (ii) chloride ion, and (iii) potassium ion.

As used herein, the term “ammonium ion” refers to the cation NH4⁺. It is formed by the protonation of ammonia (NH₃). The ammonium cation is found in a variety of salts such as ammonium carbonate, ammonium chloride, and ammonium bicarbonate. Most simple ammonium salts are very soluble in water. The ammonium ion is generated when ammonia, a weak base, reacts with Brønsted acids (proton donors) such as water: H⁺+NH₃→NH₄⁺.

According to preferred embodiments of the invention, the source of ammonium ion is a soluble salt. Suitable soluble ammonium salts and compounds include, but are not limited to, ammonium chloride, ammonium citrate (mono and dibasic), ammonium phosphate (mono and dibasic), and ammonium carrageenan. In a preferred embodiment the salt is ammonium chloride.

As used herein, the term “potassium ion” refers to the cation K⁺. The potassium ion is colorless in water and may be formed when a compound such as potassium chloride is dissolved in water or other polar solvents. According to preferred embodiments of the invention, the source of potassium ion is a soluble salt. Suitable soluble potassium salts include potassium chloride, potassium gluconate, potassium citrate. In a preferred embodiment the salt is potassium chloride.

As used herein, the term “chloride ion” refers to the anion Cl⁻. It is formed when the element chlorine gains an electron or when a compound such as hydrogen chloride is dissolved in water or other polar solvents. Chloride salts such as sodium chloride are often very soluble in water. According to preferred embodiments of the invention, the source of chloride ion is a soluble salt. Suitable soluble chloride salts include, but are not limited to, ammonium chloride, potassium chloride, sodium chloride, magnesium chloride. In a preferred embodiment the salt is a combination of ammonium chloride, potassium chloride, and sodium chloride.

Ammonium ion is required for addressing alkalosis because it provides a source of acid (H+ ion) able to be efficiently absorbed across the gastrointestinal tract and into the body, unlike other sources of acid, including strong acids (such as hydrochloric acid, HCl) and weak acids (such as acetic acid, CH₃COOH), which contribute mainly to the acidity of the stomach contents and are less efficient at contributing to plasma H+. Ammonium ion is metabolized in the liver to provide H+ ions, which are then directly available to acidify the blood and other body fluids Ammonium chloride is generally preferred because it is rapidly absorbed, does not contribute to the CO₂ load (unlike ammonium carbonate and ammonium bicarbonate), and is clinically proven to be safe and effective at lowering blood pH in oral and intravenous formulations, in infants, children, and adults. Furthermore, ammonium chloride is preferred as it provides chloride ion. Chloride ion helps to reverse alkalosis by acting on the kidney to stimulate the excretion of bicarbonate. This makes ammonium chloride, which provides both ammonium ion and chloride ion, an ideal candidate to address alkalosis.

Potassium is another key ingredient required to restore optimal blood pH according to the principles of the present invention. Indeed, serum potassium levels drop during alkalosis as potassium shifts intracellularly (into cells) in response to high serum pH. In addition, under conditions of physiological stress, catecholamines (such as the stress hormone epinephrine) also cause an intracellular potassium shift, further lowering serum potassium levels. Restoring normal serum potassium levels also contributes to reversing clinical alkalosis, because potassium is essential for the kidney to excrete excess alkali.

Chloride ion is required for addressing alkalosis because chloride induces bicarbonate excretion by the kidney. In preferred embodiments the chloride ion is provided simultaneously with potassium and ammonium by using suitable soluble salts (e.g. potassium chloride, ammonium chloride). In some embodiments, a distinct soluble chloride salt is used (e.g. sodium chloride, magnesium chloride, calcium chloride). A combination of chloride containing salts is generally preferred because this increases the total available amount of chloride supplied, while concomitantly providing useful cations such as the above mentioned ammonium and potassium, and such as sodium, which is useful in addressing dehydration (see hereinafter).

In embodiments, the composition comprises: about 0.006% w/v to about 0.07% w/v of ammonium ion, preferably about 0.01% w/v to about 0.04% w/v of ammonium ion, more preferably about 0.02% w/v to about 0.03% w/v of ammonium ion; about 0.04% w/v to about 0.9% w/v of chloride ion, preferably about 0.06% w/v to about 0.5% w/v of chloride ion, more preferably about 0.1% w/v to about 0.4% w/v of chloride ion; and about 0.01% w/v to about 0.15% w/v of potassium ion, preferably about 0.02% w/v to about 0.1% w/v of potassium ion, more preferably about 0.03% w/v to about 0.05% w/v of potassium ion.

The composition of the invention may also comprises further components useful for treating and/or preventing alkalosis, including but not limited to hydrochloric acid, hydrogen ion, and compounds which can release acids once metabolized such as lysinemonohydrochloride, and combinations thereof.

According to a further aspect, the edible or potable compositions of the invention aim to prevent or treat dehydration (e.g. achieve or maintain a desirable state of hydration) caused by airline travel and high altitude.

Unlike athletes who lose electrolytes through perspiration or patients who may require rehydration due to vomiting or diarrhea, airline passengers lose water mainly through insensible perspiration and by respiration in the very dry cabin air. Therefore airline passengers lose proportionally far more water than electrolytes.

Accordingly, the composition of the invention may further comprise one or more ingredients which have been selected for treating or preventing dehydration caused by airline travel and/or high altitude. In one embodiment, the one or more ingredients for addressing dehydration are selected from sodium ion and sugars.

In preferred embodiments, the composition further comprises sodium ion. As used herein, the term “sodium ion” refers to the cation Na+. The sodium ion may be formed when a compound such as sodium chloride is dissolved in water or other polar solvents. According to preferred embodiments of the invention, the source of sodium ion is a soluble salt. Suitable soluble sodium salts include, but are not limited to, sodium chloride, sodium citrate (e.g. mono sodium citrate, disodium citrate, trisodium citrate, anhydrous, dihydrate, sesquihydrate, etc.), sodium ascorbate, or any other suitable salt containing sodium. In a preferred embodiment the salt is sodium chloride.

In embodiments, the composition further comprises glucose and/or galactose as the sugar. The glucose and/or galactose may be supplied as such or as disaccharides containing glucose and/or galactose, including but not limited to lactose (glucose-galactose), sucrose (glucose-fructose), trehalose (glucose-glucose), maltose (glucose-glucose), isomaltulose (glucose-fructose), and combinations thereof.

In embodiments, the amount of sodium ion and sugars is adjusted according to the desired use. Because airline passengers require a low solute (e.g. sodium and sugar) rehydration formulation compared with dehydrated patients or athletes, the composition is preferably formulated for comprising lower concentrations of sodium and sugar than what is typically supplied in “sports” drinks or supplied by the World Health Organization in first aid oral rehydration formulations.

In a preferred embodiment, the composition is formulated for optimal absorption of water and, to achieved this, sodium and glucose are supplied in molar ratios of sodium:glucose between 2:1 and 1:1.

In a preferred embodiment for airline travel, the composition comprises about 0.01% w/v to about 0.03% w/v of sodium ion. In a preferred embodiment for adaptation to altitude with moderate exercise (such as walking and sightseeing), the composition comprises about 0.04% w/v to about 0.1% w/v of sodium ion. In a preferred embodiment for adaptation to altitude with strenuous exercise (such as trekking or climbing), the composition comprises about 0.04% w/v to about 0.3% w/v of sodium ion.

In a preferred embodiment for airline travel, the composition comprises about 0.1% w/v to about 0.3% w/v of glucose and/or galactose. In a preferred embodiment for adaptation to altitude with moderate exercise (such as walking and sightseeing), the composition comprises about 0.3% w/v to about 2% w/v of glucose and/or other simple carbohydrates. In a preferred embodiment for adaptation to altitude with strenuous exercise (such as trekking or climbing), the composition comprises about 1% w/v to about 6% w/v of glucose and/or other simple carbohydrates.

Although not directed specifically to sports-related dehydration, compositions of the invention may be suitable for athletes performing at high altitude. Accordingly, the solute concentrations of the composition may be adjusted to provide higher concentrations of sugars to replenish energy, and higher concentrations of electrolytes to replace those lost through perspiration.

According to a further aspect, the edible or potable compositions of the invention aim to promote optimal immune system function during flights and/or at high altitude, as well as to alleviate immune suppression caused by airline travel and high altitude.

The term “promote immune function”, “promote immunity” “alleviate immune suppression” and related terms, refer to an improvement of immune function(s) leading to a better health condition and/or a decreased risk of contracting infectious diseases. Particular examples may include, but are not limited to, an increase in serum values of immune protective molecules, an increase in indicators of immune function such as natural killer cell count, natural killer cell activity, lymphocyte cell count, lymphocyte activity, protective serum antibodies, immune barrier integrity, or phagocytic activity, and/or a decrease in harmful immune activity including markers of inflammatory disease and signs and symptoms of allergy, asthma, and autoimmune disease.

Accordingly, the compositions of the invention may further comprise an ingredient to promote immune function. In various embodiments, the ingredient to promote immune function is selected from ascorbic acid, zinc, vitamin D3, L-glutamine, L-tryptophan, 5-hydroxytryptophan and combinations thereof.

Ascorbic acid, an easily absorbed form of vitamin C, is well established to contribute to immune function. It may contribute to the health and repair of tissues involved in bather immunity, which tend to be damaged by the extremely dry environment of air travel and high altitude. In one embodiment, the composition comprises about 0.005% w/v to about 0.1% w/v of ascorbic acid, preferably about 0.05% w/v to about 0.1% w/v of ascorbic acid. Ascorbic acid can also be provided as ascorbate salts, including but not limited to sodium ascorbate, calcium ascorbate, magnesium ascorbate.

Zinc is known to be important for proliferation of immune cells and for wound healing. Furthermore, zinc can block attachment and invasion of rhinoviruses, a causative factor of upper respiratory tract infections including the common cold, into epithelial cells lining the nose and throat. Providing zinc during air travel could support cellular immune function, immune barrier integrity, and directly inhibit infection by viruses. In one embodiment, the composition comprises about 0.0003% w/v to about 0.002% w/v of zinc, preferably about 0.001% w/v to about 0.002% w/v of zinc.

Vitamin D is also known to have immune enhancing properties. The preferred form of vitamin D for supplementation is vitamin D3 (cholcalciferol). Vitamin D may also be supplied as vitamin D2 (ergocalciferol). In one embodiment, the composition comprises about 5×10⁻⁷% w/v to about 4×10⁻⁶% w/v of vitamin D3 (i.e. about 5 μg/l to about 40 μg/l), preferably about 2×10⁻⁶% w/v to about 4×10⁻⁶% w/v (preferably about 20 μg/l to about 40 μg/l).

L-glutamine (Chemical Abstract Society (CAS) Database Reference 56-85-9) and L-tryptophan (CAS #73-22-3) are amino acids that stimulate the proliferation of immune cells, and could therefore support immune function. L-glutamine tends to boost energy levels, while L-tryptophan and its metabolite 5-hydroxytryptophan tend to promote restful sleep and relaxation. Because L-tryptophan is poorly soluble in water whereas 5-hydroxytryptophan has a high solubility of 10 g/L, 5-hydroxytryptophan is preferable for use in a beverage. In one embodiment, the composition comprises about 0.01% w/v to about 0.2% w/v of L-glutamine, preferably about 0.1% w/v to about 0.2% w/v of L-glutamine. In one embodiment, the composition comprises about 0.001% w/v to about 0.02 w/v of 5-hydroxytryptophan, preferably about 0.01% w/v to about 0.02% w/v of 5-hydroxytryptophan.

In other embodiments, the compositions of the invention may further comprise additional ingredient(s), including but not limited to stimulants, relaxants, stabilizers, coloring agents, vitamins, acidifying agents, preservatives, sweeteners, flavorings etc.

In one embodiment, the composition further comprises flavoring agent(s) that may also have immune benefits and/or other beneficial properties. Examples of envisioned flavoring agents include, but are not limited to, green tea extracts, ginger extracts, chamomile extracts, elderberry extracts, pomegranate extracts, blueberry extracts, cranberry extracts, citrus fruit extracts, and mixtures thereof. As used herein, the term “extract” encompasses functionally equivalent infusions, solutions, concentrates, and powders.

In one embodiment, the composition further comprises coloring agent(s) that may also have immune benefits and/or other beneficial properties. Examples of envisioned coloring agents include, but are not limited to, turmeric extract, green tea extract, elderberry extract, pomegranate extract, blueberry extract, and mixtures thereof. For commercial preparations, the choice and amounts of the flavor/color ingredients may be adjusted according to the market.

In one embodiment the composition further comprises a stimulant. Examples of envisioned stimulants include, but are not limited to, L-glutamine, green tea extract including compounds derived from green tea such as L-theanine, rooibos extract, vitamin B, or any combination of any two or more thereof. In preferred embodiments, the stimulant is L-glutamine.

In one embodiment the composition further comprises an agent to promote relaxation and/or to decrease anxiety (e.g. anxiolytic agent). Examples of envisioned relaxants/anxiolytic agents include, but are not limited to, L-tryptophan, 5-hydroxytryptophan, chamomile, or any combination thereof. Preferably, the relaxant is 5-hydroxytryptophan.

In one embodiment the composition further comprises a blend of herbal concentrates and extracts, and fruit juice concentrates and extracts. Preferably, the blend of herbal concentrates and extracts and fruit juice concentrates and extracts promotes immune function. Examples of envisioned herbal concentrates/extracts and fruit juice concentrates include, but are not limited to, green tea extracts, ginger extracts, chamomile extracts, elderberry extracts, pomegranate extracts, blueberry extracts, cranberry extracts, citrus fruit extracts, and mixtures thereof.

In one embodiment, the composition further comprises a stabilizer. Examples of envisioned stabilizers include, but are not limited to, antifoaming agents, malic acid, citric acid, potassium sorbate, sodium benzoate, and mixtures thereof.

According to some embodiments, the composition may further comprise magnesium ion and/or calcium ion. Indeed, magnesium may help to restore normal potassium serum levels after potassium depletion. Magnesium may also synergize with potassium in attenuating stress. Regarding calcium, it is known that calcium serum levels drop during alkalosis. Supplying calcium could help to prevent or reverse this process, and may synergize with potassium in preventing muscle cramping. In addition, although is not a primary objective of the present invention, the composition may also provide a clinically relevant calcium supplement (e.g. for the prevention of osteoporosis).

As used herein, the term “magnesium ion” and “calcium ion” refers to the cations Mg²⁺ and Ca²⁺, respectively. The magnesium and calcium ions may be formed when compounds such as magnesium chloride and calcium chloride are dissolved in water or other polar solvents. According to preferred embodiments of the invention, the source of magnesium and calcium ions is a soluble salt. Examples of envisioned soluble magnesium salts include, but are not limited to, magnesium chloride, magnesium citrate, magnesium ascorbate, magnesium gluconate, magnesium malate, magnesium glycinate. Examples of envisioned soluble calcium salts include, but are not limited to, calcium chloride, calcium citrate, calcium ascorbate, calcium gluconate, calcium carbonate, tricalcium phosphate. In preferred embodiments the magnesium salt is magnesium chloride and the calcium salt is calcium chloride.

According to some embodiments, the composition may further comprise vitamins and/or minerals. The amounts of vitamins and minerals to be used are preferably those typical of liquid nutritional formulations known to those skilled in the art. Examples of envisioned vitamins and/or minerals include, but are not limited to, vitamin B complex, vitamin C, Vitamin D, calcium, magnesium, zinc, selenium compounds.

According to some embodiments, the composition may further comprise an acidifying agent. Acidifying agents may be particularly useful for maintaining a liquid beverage according to the invention at a predetermined pH. Examples of envisioned acidifying agents include, but are not limited to, malic acid, citric acid, and mixtures thereof.

According to some embodiments, the composition may further comprise a preservative. Examples of envisioned preservatives include, but are not limited to, potassium sorbate, sodium benzoate, and mixtures thereof.

According to some embodiments, the composition may further comprise fructose or other sugars or carbohydrates as a source of energy or as an additional sweetener. According to some embodiments, the composition may further comprise additional ingredients to enhance the beverage by masking the taste of the active ingredients and adding desirable food value, such as milk, flavored milk, chocolate milk, almond milk, soy milk, etc in liquid forms or as dehydrated powders. For use during exercise at high altitude, the composition may further comprise protein-containing formulation (such as whey protein, soy protein). In addition for use at high altitude, the formulation may contain adaptogens which facilitate or accelerate the acclimatization to high altitude or protect against the effects of hypoxia, such as vitamins B2, B6, carnitine, carnosine, N-acetyl-cysteine, selenium, rhodiola, and the like. In addition for use at high altitude during exercise, additional additives to support high performance physical exercise may also be included. In particular, as muscle metabolism generates ammonium, ingredients to support ammonia metabolism (such as citrulline, alpha-ketoglutarate, and the like) may be useful additives to counter the combination of metabolically derived ammonium and ammonium contained in the composition of the invention.

B) Methods of preparation. In general, the compositions according to the present invention may be prepared by any conventional methods, using readily available and/or conventionally preparable starting materials, reagents and conventional synthesis procedures.

Preferably the ingredients are combined in a way that reduces any undesirable tastes or textures associated with mixing the ingredients, while maximizing the effectiveness of each of the ingredients entering into the final composition.

The composition may be formulated as an aqueous solution (e.g. beverage) or as a powder, soluble or effervescent tablet, paste or gel for later dissolution in a suitable aqueous solution (e.g. water (e.g. purified or distilled), juice, tea, herbal tea or infusion, flavored water, vitamin water, carbonated beverages, milk, flavoured milk, chocolate milk, soy milk, almond milk, drinkable yogurt, protein drink, energy drink, sports drink, alcoholic beverages and the like). The composition may be formulated as a ready to use liquid beverage or as a liquid concentrate (e.g. 2×, 3×, 4×, 5×, 10× etc.) for further dilution. Preferably the composition is formulated for human consumption (e.g. pleasant taste, suitable dosage, etc.).

For a composition formulated as a liquid, solid and/or liquid ingredients may be mixed with a predetermined volume of filtered or distilled water. The resulting mixed solution may be adjusted to the desired pH by addition of suitable acidifying agents, it may be colored or flavored, sweetened, etc. as appropriate.

For a composition formulated as a solid, the composition may be prepared by mixing suitable powders providing each of the desired ingredients. The suitable powders may be obtained as is or be prepared by drying liquids, such as through spray drying or lyophilization. The powders may be ground to obtain final solid composition having a fine or coarse texture. The final solid composition may be packaged to contain a single dose (e.g. a small pouch) or to contain a plurality of doses for consumption (a bulk container of multiple servings such as 100 doses). The solid ingredients may further be packaged or incorporated into pastes, gels, concentrates, tablets, effervescent tablets, capsules, or the like for dissolution into beverages and/or addition to food by the manufacturer or consumer, and/or for consumption as-is by the consumer.

Preferably, in a composition formulated as a liquid concentrate or as a powder, the amount of each ingredient is dosed such that, when the concentrate or powder is dissolved or suspended for consumption, the final concentration of the ingredients will be as recited herein.

According to a preferred embodiment, the invention relates to a potable liquid beverage. In one particular embodiment, the potable liquid beverage comprises the following ingredients per 1,000 ml: ammonium ion about 60 mg to about 750 mg (preferably about 125 mg to about 375 mg, more preferably about 185 mg to about 280 mg); chloride ion about 250 mg to about 9,000 mg (preferably about 600 mg to about 5,000 mg, more preferably about 900 mg to about 4,500 mg); potassium ion about 100 mg to about 1,500 mg (preferably about 200 mg to about 1,000 mg, more preferably about 300 mg to about 500 mg); sodium ion about 70 mg to about 2,600 mg (preferably for airline travel about 70 mg to about 350 mg, more preferably for airline travel about 115 mg to about 175 mg, preferably for exercise at high altitude about 200 mg to about 2,600 mg, more preferably for exercise at high altitude about 400 mg to about 900 mg); glucose and/or galactose about 900 mg to about 60,000 mg (60 g) (preferably for airline travel about 900 mg to about 2,700 mg, more preferably for airline travel about 900 mg to about 1,400 mg, preferably for exercise at high altitude about 10,000 mg (10 g) to about 60,000 mg (60 g)); water to make 1,000 ml.

According to one particular embodiment, the potable liquid beverage further comprises about 40 mg/l to about 400 mg/l of magnesium ion and/or about 50 mg/l to about 500 mg/l of calcium ion, preferably for airline travel about 40 mg/l to about 400 mg/l of magnesium ion and about 50 mg/l to about 200 mg/l calcium ion, and preferably for exercise at high altitude about 40 mg/l to about 400 mg/l of magnesium ion and about 100 mg/l to about 500 mg/l calcium ion.

According to one particular embodiment, the potable liquid beverage further comprises about 50 mg/l to about 1,000 mg/l of ascorbic acid, preferably about 500 mg/l to about 1,000 mg/l ascorbic acid.

According to one particular embodiment, the potable liquid beverage further comprises about 3 mg/l to about 20 mg/l of zinc ion, preferably about 10 mg/l to about 20 mg/l zinc ion.

According to one particular embodiment, the potable liquid beverage further comprises about 100 mg/l to about 1,500 mg/l of L-glutamine, preferably about 750 mg/l to about 1,500 mg/l of L-glutamine.

According to one particular embodiment, the potable liquid beverage further comprises about 10 mg/l to about 150 mg/l of 5-hydroxytryptophan, preferably about 75 mg/l to about 150 mg/l of 5-hydroxytryptophan.

Preferably the ions are soluble and they derive from the solubilisation of water soluble salts.

Those skilled in the art will readily understand that the present invention is not limited to a particular volume. A volume of 1,000 ml as provided above is one of many possible examples. According to one embodiment, a volume of 1,000 ml may constitute a single serving or it may be divided into smaller servings of 250 ml, 500 ml, 750 ml. Larger volumes or servings (e.g. 1.25 l, 1.5 l, 1.75 l, 2 l, etc.) may also be envisioned. According to a preferred embodiment, a serving is about 500 ml. Similarly, a skilled person will know how to adjust the abovementioned value to manufacture a liquid concentrate that may be diluted to desirable values.

A liquid beverage according to the invention may be packaged in any suitable container, including but not limited to plastic bottles (reclosable or not), aluminum cans, aseptic carton packages such as Tetra Brik™, etc. Aseptic containers may be preferred in order to give more shelf life and the possibility of room temperature storage. However, any other liquid packaging know to those skilled in the art may be suitable.

According to a particular embodiment, the composition of the invention is formulated in a solid form (e.g. a powder). A composition in a solid form of the invention may be packaged in any suitable container, including but not limited to plastic bottles (reclosable or not), aluminum cans, foil and/or plastic packages, pouches, spout pouches, sachets, packets, cartons or bags (reclosable or not), bulk containers, canisters etc. or may further be packaged or incorporated into pastes, gels, concentrates, tablets, effervescent tablets, capsules, or the like for dissolution into beverages and/or addition to food by the manufacturer or consumer, and/or for consumption as-is by the consumer.

In one embodiment (e.g. a formulation consisting almost entirely of active ingredients with minimal flavorings, sweeteners, and the like), the composition comprises about 5 grams of solid (e.g. a single-serving pouch or packet), for dilution by the consumer into about 500 ml water (or other suitable aqueous liquid), in order to provide 1 serving of beverage. In a preferred embodiment the composition comprises the following ingredients per 5 gram of solid: ammonium ion about 50 mg to about 200 mg; chloride ion about 300 mg to about 2,500 mg; potassium ion about 100 mg to about 500 mg; sodium ion about 35 mg to about 175 mg; glucose and/or galactose about 500 mg to about 1,350 mg; additional ingredients and/or excipient(s) to make 5 g.

In one embodiment (e.g. a sweetened, flavoured composition, which may contain large amounts of additional sugars, protein powders, milk powders, and the like, containing an effective quantity of active ingredients per serving, but wherein active ingredients constitute a lower percentage of total ingredients than the minimal preparation described above due to the large amount of additional ingredients), the composition comprises about 100 grams of solid (e.g. a single-serving pouch or packet), or a larger reclosable container such as a bottle or spout pouch containing powder, to which the consumer may directly add liquid), for dilution by the consumer into about 500 ml water (or other suitable aqueous liquid), to provide 1 serving of beverage. In a preferred embodiment the composition comprises the following ingredients per 100 gram of solid: ammonium ion about 50 mg to about 200 mg; chloride ion about 300 mg to about 2,500 mg; potassium ion about 100 mg to about 500 mg; sodium ion about 35 mg to about 1,300 mg; glucose and/or galactose about 500 mg to about 50 g; additional ingredients and/or excipient(s) to make 100 g.

According to one particular embodiment consisting almost entirely of active ingredients, the solid comprises about 30 mg/g to about 120 mg/g of ammonium chloride and about 40 mg/g to about 200 mg/g of potassium chloride as a source of ammonium, chloride and potassium ions. According to one particular embodiment comprising a sweetened and/or flavoured composition, the solid comprises about 1 mg/g to about 6 mg/g of ammonium chloride and about 2 mg/g to about 10 mg/g of potassium chloride as a source of ammonium, chloride and potassium ions.

According to one particular embodiment consisting almost entirely of active ingredients, the solid comprises about 20 mg/g to about 90 mg/g of sodium chloride as a source of sodium ion. According to one particular embodiment comprising a sweetened and/or flavoured composition, the solid comprises about 1 mg/g to about 32 mg/g of sodium chloride as a source of sodium ion.

According to one particular embodiment consisting almost entirely of active ingredients, the solid further comprises about 4 mg/g to about 40 mg/g of magnesium ion and/or about 4 mg/g to about 20 mg/g calcium ion. According to one particular embodiment comprising a sweetened and/or flavored composition, the solid further comprises about 0.2 mg/g to about 2 mg/g of magnesium ion and/or about 0.5 mg/g to about 2 mg/g calcium ion. According to one particular embodiment consisting almost entirely of active ingredients, the solid comprises 16 mg/g to about 160 mg/g of magnesium chloride a source of magnesium ion. According to one particular embodiment comprising a sweetened and/or flavored composition, the solid comprises about 1 mg/g to about 8 mg/g of magnesium chloride as a source of magnesium ion. According to one particular embodiment consisting almost entirely of active ingredients, the solid comprises about 14 mg/g to about 60 mg/g of calcium chloride a source of calcium ion. According to one particular embodiment comprising a sweetened and/or flavoured composition, the solid comprises about 1 mg/g to about 7 mg/g of calcium chloride a source of calcium ion.

According to one particular embodiment consisting almost entirely of active ingredients, the solid further comprises about 10 mg/g to about 150 mg/g of L-glutamine, preferably about 60 mg/g to about 150 mg/g of L-glutamine. According to one particular embodiment comprising a sweetened and/or flavoured composition, the solid further comprises about 0.5 mg/g to about 7 mg/g of L-glutamine, preferably about 3 mg/g to about 7 mg/g of L-glutamine.

According to one particular embodiment consisting almost entirely of active ingredients, the solid further comprises about 2 mg/g to about 16 mg/g of 5-hydroxytryptophan. According to one particular embodiment comprising a sweetened and/or flavoured composition, the solid further comprises about 0.1 mg/g to about 1 mg/g of 5-hydroxytryptophan, preferably about 0.5 mg/g to about 1 mg/g of 5-hydroxytryptophan.

Preferably the ions are provided as soluble salts.

According to one particular embodiment, the solid further comprises one or more additional ingredients selected from flavoring agents, coloring agents, sweeteners, anti-caking ingredients (e.g. calcium aluminum silicate, calcium phosphate tribasic, calcium silicate, calcium stearate, magnesium carbonate, magnesium silicate, magnesium stearate, silicon dioxide, sodium aluminum silicate), excipients and combinations thereof.

A container comprising a composition in concentrate form, solid form and/or tablet, gel, paste or powder form as defined herein may further comprise a label with instructions for dilution or dissolution of the concentrate or solid or tablet or gel or paste or powder into a potable aqueous liquid.

The compositions of the invention (as a liquid or solid form) may be provided as part of a kit. For instance the kit may comprise a plurality of individual doses ready for consumption (e.g. different formulations comprising various flavors or comprising various concentrations or combinations of the ingredients for different uses). The kit may also comprise the composition under different forms (e.g. ready to drink liquid(s), ready to eat snacks, paste(s), gel(s), liquid concentrate(s), tablet(s), effervescent tablet(s), capsule(s), or the like, in pouch(es), spout pouch(es), packet(s), can(s), bottle(s), powder container(s), etc.).

In one particular embodiment the kit comprises: (i) at least two of an edible or potable composition as defined herein; (ii) at least two containers as defined herein; and (iii) a combination of (i) and (ii).

In another particular embodiment the kit comprises instructions for using the composition of the invention to alleviate undesirable physiological effects caused by airline travel and/or exposure to high altitude.

Related aspects of the invention concern methods of uses for treating alkalosis, hypoxia and/or dehydration, and more particularly uses of compositions as defined herein for alleviating undesirable physiological effects caused by airline travel and/or exposure to high altitude.

According to one particular aspect, the invention relates to a method for alleviating undesirable physiological effects caused by airline travel and/or exposure to high altitude (e.g. over about 1,500 meters above sea level, preferably over 2,400 meters above sea level), the method comprising the steps of: providing an edible or potable composition or a potable liquid beverage and/or a powder, paste, gel, concentrate, tablet, effervescent tablet, capsule, or the like as defined herein; and orally administering said composition, liquid beverage and/or powder, paste, gel, concentrate, tablet, effervescent tablet, capsule, or the like to a human subject in need thereof.

The administering may comprise orally administering concomitantly a volume of a potable aqueous liquid (e.g. water, juice, etc.). As used herein, the term “concomitant” or “concomitantly” as in the phrases “administering concomitantly” or “concomitantly with” includes administering a first agent in the presence of a second agent. A concomitant administration includes methods in which the first or second agents are co-administered. A concomitant administration treatment method may be executed step-wise by different actors. For example, one actor may administer to a subject a first agent and as a second actor may administer to the subject a second agent. The administering steps may be executed at the same time, or nearly the same time (e.g. within 1, 2, 5, 10, 20 or 30 minutes), or at distant times (e.g. more than 1 hour). The actor and the subject may be the same entity (e.g., a human).

Preferably, the edible or potable composition and/or the powder is dissolved into the potable aqueous liquid for oral administration (e.g. drinkable beverage). Such dissolution may be carried out at the time or shortly before administration. The dissolution may also have been done in the past (e.g. as a ready-to-drink commercially available beverage).

According to one embodiment, the method comprises administering the composition for about 2 hours to about 96 hours.

According to one particular embodiment wherein the composition is used for alleviating undesirable physiological effects caused by airline travel, the composition is administered for about 2 hours to about 24 hours (e.g. one individual dose every 2-4 hours, or every 3-6 hours, or every 4-8 hours, depending on the activity level and thirst of the subject, e.g. if the subject is awake or asleep).

According to one particular embodiment wherein the composition is used for alleviating undesirable physiological effects caused by exposure to altitude, the composition is administered for about 2 hours to about 96 hours (e.g. one individual dose every 2-4 hours, or every 3-6 hours, or every 4-8 hours, etc. depending on factors such as the altitude, the activity level of the subject, the size and age of the subject, whether the subject is awake or sleeping, time allowed or required for acclimation to a given altitude and/or depending on whether altitude is constant, increasing and/or diminishing over a given time period).

In various embodiments, administration of a composition according to the invention will results in one or more of the followings: decreasing a serum pH of above about 7.45 to a physiologically normal range of about 7.37 to about 7.45; maintaining a serum pH in a physiologically normal range of about 7.37 to about 7.45; preventing or reducing undesirable physiological effects associated with respiratory alkalosis; increasing the oxygen dissolved in the blood (PO₂); decreasing the CO₂ dissolved in the blood (PCO₂); increasing an O₂ saturation level to a normal value of about 95-97%; maintaining an O₂ saturation level at an optimal value of about 97-99%; increasing or maintaining extracellular fluid volume or decreasing hematocrit that was increased due to hypovolemic dehydration; increasing serum values of ascorbic acid, vitamin D3 and/or zinc; increasing one or more of natural killer cell count, natural killer cell activity, lymphocyte cell count, lymphocyte activity, serum antibodies, and phagocytic activity; and decreasing serum markers, signs, or symptoms of inflammation, allergic, or autoimmune disease activity.

Those skilled in the art will appreciate the benefits of the compositions according to the invention and may readily identify additional uses. For instance the compositions and beverages according to the invention may be beneficial to airline crew and others performing work during air travel or in flight; military personnel engaged in operating or crewing airborne vehicles and/or being deployed by air; professional or recreational athletes needing to acclimate rapidly to altitudes above sea level in order to maximize performance; military personnel being deployed temporarily or without prior acclimatization to high altitude; any individual allergic to or otherwise unable to tolerate acetazolamide (“Diamox™”), prescribed to prevent and treat Acute Mountain Sickness; as a replacement for acetazolamide (which is on the World Health Organization's list of Essential Medicines) for situations where a gentler, less expensive, or non-prescription alternative is desired to a prescription medicine with multiple side effects; animals brought temporarily to high altitude in order to perform athletically or to support human activities (e.g. race horses, rescue dogs, military working dogs, service animals, companion animals); as an energy drink or performance enhancer specifically designed for high altitude; as a sleep aid for use in flight, at high altitude, and in people with sleep disorders such as sleep apnea; in conditions of low ambient air pressure and/or crowding for example space stations, emergency shelters sealed to outside atmosphere, etc.; in certain cases of metabolic alkalosis, particularly in contraction alkalosis (dehydration) and in people who have been vomiting; in situations where increased ventilation could contribute to increased alertness such as to counter sleepiness in people operating vehicles, or as a study aid; in free divers as pre-dive preparation to reduce the partial pressure of carbon dioxide (PCO₂) in the blood and increase the partial pressure of oxygen (PO₂) in the blood; in situations where increased ventilation could decrease discomfort or pain such as for treatment of hangovers, headaches and migraines.

### EXAMPLES

### Example 1: Calculations of a Potentially Useful Dosage of Ammonium Chloride for Treating Alkalosis in Commercial Airline Passengers

Those skilled in the art will appreciate that dosages and effective amounts of each of the ammonium ions, chloride ions and potassium ions entering into the composition of the invention may vary for example, depending upon a variety of factors including the compound employed, the age, body weight, general health, gender, and diet of the subject, the time of administration, the rate of excretion, any drug combination, if applicable, and the particular use or effect desired. In addition, the most effective amount may depend on the subject's blood parameters (e.g., calcium levels, lipid profile, insulin levels, glycemia), an existing disease state, etc. Such appropriate doses may be determined using any available assays including the assays described herein. When the composition of the invention is to be administered to humans, the subject may choose to take a relatively low dose at first, subsequently increasing the dose until an appropriate response is obtained.

For instance, a potentially useful dosage of ammonium chloride can be calculated by estimating the base excess caused by increased ventilation triggered by low cabin pressure (or low air pressure at altitude). Multiple studies concur that O₂ hemoglobin saturation in airline passengers drops by 5-6%, from a normal value of 98-99% on the ground down to 92-94% at cruising altitude. This level of oxygen saturation, for a partial pressure of O₂ corresponding to the normal cabin pressurization of 8,000 feet (574 mm Hg), indicates that respiratory compensation has taken place. Uncompensated breathing at 574 mm Hg would result in 60 mm Hg O₂ partial pressure in the blood, and 91% O₂ saturation, based on standard physiological tables. Therefore, to achieve 93% O₂ saturation at 574 mm Hg, 66-68 mm Hg partial pressure of O₂ in the blood is required. This in turn implies an increase in ventilation rate from 14/minute to 16/minute on average (with no change in tidal volume). This increase in ventilation would cause a corresponding decrease in CO₂ partial pressure in the blood to 32 mm Hg (normal 35-45 mm Hg).

Using the Henderson-Hasselbalch equation, the blood pH of an airline traveler in a cabin pressurized to 8,000 feet can be estimated as follows: pH=pK(CO₂)+log ([HCO₃⁻]/CO₂solubility*PCO₂)=6.1+log (24/0.03*32)=7.50 where pKa(CO₂) is a constant=6.1 at 37° C. (body temperature), [HCO₃⁻] is taken to be 24 mM (normal range=22-26 mM), CO₂ solubility is a constant=0.03, and PCO₂ is the partial pressure of CO₂ calculated above at 32 mm Hg.

The range of blood pH considered clinically normal is 7.35 to 7.45, more preferably 7.38 to 7.42. Therefore, the estimated blood pH of 7.50 represents significant clinical alkalosis and deserves treatment.

The base excess corresponding to a blood pH of 7.50 can be calculated using the standard equation, base excess (mEq/L)=0.93×([HCO₃⁻]−24.4+14.8×(pH −7.4))

Assuming normal initial uncompensated bicarbonate blood levels of 24 mM as above (normal range=22-26 mM), base excess=0.93×(24-24.4+14.8×(7.5−7.4))=1.0044 mEq/L

If the blood pH in airline passengers is moderated by the abnormally high ambient CO₂ in the cabin, blood pH may not rise as much as the above estimate suggests. Assuming the maximum published level of 2.6% for cabin CO₂ the above equations yield a blood pH of 7.46, which in turn corresponds to an estimated base excess of 0.4538 mEq/L.

Using the averaged base excess from the above calculations, and the following standard equation, total base excess (mEq)=base excess (mEq/liter)×0.3 (liter/kg)×body weight (kg), then we arrive at a base excess of 15.3 mEq for an average 70 kg man traveling in an airline cabin pressurized to 8,000 feet. Clinical recommendations for correcting alkalosis with intravenous HCl suggest supplying ⅔ of the acid equivalents over 1 to 2 hours: 15.3 mEq×⅔=10.3 mEq which corresponds to 551 mg NH₄Cl.

Therefore 551 mg ammonium chloride should largely prevent or reverse the alkalosis caused by short-haul air travel (3 hours or less of flying time). Preferably this amount of ammonium chloride should be divided into two equal servings. For optimal effectiveness, the first serving should be consumed as early as possible during the flight, or during the pre-boarding wait, and the second serving may be consumed slowly over the remainder of the flight. Treatment with ammonium chloride should prevent or reduce alkalosis, in turn preventing or reducing the downstream metabolic disturbances caused by alkalosis, such as drops in serum potassium and calcium. In addition, treatment with ammonium chloride should cause a sustained increase in the respiratory rate (ventilation), which enhances the concentration of oxygen dissolved in the blood, and maximizes the O₂ hemoglobin saturation possible at any given atmospheric pressure. Together, these enhanced blood oxygen levels should improve tissue oxygenation, resulting in subjective feelings of increased energy and well-being, and decreased fatigue and lethargy.

For longer flights, an additional serving of beverage should be consumed approximately every 2-4 hours for the duration of the flight, as respiratory alkalosis is a process which will continue as long as the air pressure stays low, that is, as long as the plane remains in flight and/or pressurized to about 8,000 feet equivalent or less.

For adaptation to high altitude, an initial serving should be consumed while ascending to altitude or as soon as possible after arrival, and additional servings of beverage should be consumed approximately every 2-4 hours during daytime activities and as needed at night, until the subject descends to a lower altitude or physiological adaptation of body fluid pH is achieved after 72 to 96 hours at high altitude, whichever comes first.

### Example 2: Preparation of Various Liquid Beverages for Different Uses

Beverage 1: Preparation of an Energizing Metabolic Repair Drink for In-Flight Use

The purpose of Beverage 1 is to provide a beverage to treat or prevent alkalosis, ameliorate hypoxia, treat or prevent dehydration, enhance immune function, and enhance the perception of energy in a subject during and following airline travel. Consumption of the beverage may result in any one or more of prevention or reduction of respiratory alkalosis, prevention or treatment of dehydration, enhanced tissue oxygenation, prevention or reduction in the risk of acquiring infections during airline travel, decreased physiological stress, reduction in the incidence, magnitude, and duration of nausea, reduction in the incidence, magnitude, and duration of headache, increased comfort, decreased muscle cramping, twitching and stiffness, increased energy levels, reduced fatigue, increased feeling of well-being and alertness, and decreased lethargy during and after airline travel.

For Beverage 1, the ingredients selected to treat alkalosis are ammonium chloride and potassium chloride at a concentration determined to be both effective (as determined in calculation presented above), and palatable (in experimental trials on board aircraft). The ingredients to treat or prevent dehydration in flight are glucose and sodium chloride at a concentration chosen to maximize absorption of water under the conditions of minimal movement and perspiration that occur in flight. The ingredients to promote immune function are zinc chloride, ascorbic acid, and vitamin D3. The multifunctional ingredients to treat alkalosis, to help maintain a normal serum pH range, to help maintain normal serum potassium levels, and to help prevent cramping are magnesium chloride and calcium chloride. All cations for or contributing to the treatment of alkalosis (ammonium ion, potassium ion, magnesium ion, calcium ion) and prevention and treatment of dehydration (sodium ion) and enhancement of immune function (zinc ion) are supplied as chloride salts, to maximize the amount of chloride delivered to the subject, as chloride contributes to correcting alkalosis by acting on the kidney. The stimulant ingredients are L-glutamine and green tea extract, which also support immune function. The ingredients used to flavor and color Beverage 1 are green tea extract and pomegranate extract. The flavor/color ingredients enhance the palatability and appearance of the beverage while also being selected from among natural and organic compounds with energizing and/or immune enhancing properties.

Beverage 1 was prepared by weighing out all the ingredients in the amounts listed in Table 1, with the exception of the flavor/color ingredients, and dissolving them in 500 ml of water. The solution was mixed gently until all ingredients were fully dissolved. The flavor/color ingredients were then adjusted to achieve the desired flavor and color, and the final volume was adjusted to 500 ml with water.

Beverage 2: Preparation of a relaxing metabolic repair drink for in-flight use. The purpose of Beverage 2 is to provide a beverage to treat or prevent alkalosis, ameliorate hypoxia, treat or prevent dehydration, enhance immune function, and promote relaxation in a subject during and following airline travel. Consumption of the beverage may result in any one or more of prevention or reduction of respiratory alkalosis, prevention or treatment of dehydration, enhanced tissue oxygenation, prevention or reduction in the risk of acquiring infections during airline travel, decreased physiological stress, reduction in the incidence, magnitude, and duration of nausea, reduction in the incidence, magnitude, and duration of headache, increased comfort, decreased muscle cramping, twitching and stiffness, increased relaxation, decreased anxiety, enhanced sleep duration and quality, reduced fatigue, increased feeling of well-being, and decreased lethargy during and after airline travel.

For Beverage 2, the ingredients to treat or prevent alkalosis are ammonium chloride and potassium chloride at a concentration determined to be both effective (as determined in calculation presented above), and palatable (in experimental trials on board aircraft). The ingredients to treat or prevent dehydration in flight are glucose and sodium chloride at a concentration chosen to maximize absorption of water under the conditions of minimal movement and perspiration that occur in flight. The ingredients to promote immune function are zinc chloride and ascorbic acid. Less ascorbic acid is included in Beverage 2 than in beverage 1 as ascorbic acid may have stimulant effects that could decrease sleep quality during flight. Similarly, vitamin D3 is omitted in this formulation as it may also alter sleep quality. The multifunctional ingredients to treat alkalosis, to help maintain a normal serum pH range, to help maintain normal serum potassium levels, and to help prevent cramping are magnesium chloride and calcium chloride. All cations required for or contributing to the prevention or treatment of alkalosis (ammonium ion, potassium ion, magnesium ion, calcium ion) and prevention and treatment of dehydration (sodium ion) and enhancement of immune function (zinc ion) are supplied as chloride salts, to maximize the amount of chloride delivered to the subject, as chloride contributes to correcting alkalosis by acting on the kidney. The anxiolytic ingredients are 5-hydroxytryptophan, magnesium, and chamomile extract. The ingredients used to flavor Beverage 2 are chamomile extract and ginger extract, as ginger also has gastrointestinal calmative effects.

Beverage 2 was prepared by weighing out all the ingredients listed in Table 1, with the exception of the flavor/color ingredients, and dissolving them in 500 ml of water. The solution was mixed gently until all ingredients were fully dissolved. The flavor/color ingredients were then adjusted to achieve the desired flavor and color and water was added to a final volume of 500 ml.

Beverage 3: Preparation of an energizing metabolic repair drink for use at high altitude during moderate to intense exercise. The purpose of Beverage 3 is to provide a beverage to treat or prevent alkalosis, ameliorate hypoxia, treat or prevent dehydration, enhance immune function, replenish electrolytes lost in sweat, provide energy in the form of simple carbohydrates, and enhance the perception of energy in a subject during acclimatization to high altitude. Consumption of the beverage may result in any one or more of prevention or reduction of respiratory alkalosis, prevention or treatment of dehydration, enhanced tissue oxygenation, decrease in periodic breathing, prevention or reduction in the risk of acquiring infections, decreased physiological stress, reduction in the incidence, magnitude, and duration of nausea, reduction in the incidence, magnitude, and duration of headache, decreased muscle cramping, twitching and stiffness, increased energy levels, reduced fatigue, increased feeling of well-being, enhanced physical performance, improved sleep quality, and decreased lethargy during adaptation to high altitude.

For Beverage 3, the ingredients to treat or prevent alkalosis are ammonium chloride and potassium chloride at a concentration determined to be both effective (as determined in calculation presented above), and palatable (in experimental trials at high altitude). The ingredients to treat or prevent dehydration at high altitude are sucrose and sodium chloride. The concentration of sucrose was determined as a compromise between supplying a low solute beverage designed to maximize absorption of water, and supplying a source of carbohydrate to replenish energy sources which are rapidly depleted during exercise, especially under the additional metabolic stressors imposed by high altitude. Sucrose was selected in preference to glucose as this glucose-fructose disaccharide will supply glucose to facilitate water absorption and therefore hydration, and fructose as an energy source that does not trigger an increase in insulin secretion, and is therefore a better compromise for ingestion during exercise. The concentration of sodium chloride was determined as a compromise between supplying a low solute beverage designed to maximize absorption of water, and supplying a source of sodium to replenish that lost during perspiration triggered by exercise. The ingredients to promote immune function are zinc gluconate, an easily absorbed form of zinc preferable for an exercising subject, and ascorbic acid supplied in amounts easily absorbable in one serving, and vitamin D3 which in addition to supporting immune function, will aid in the absorption of calcium, magnesium and zinc. The multifunctional ingredients to treat alkalosis, to help maintain a normal serum pH range, to help maintain normal serum potassium levels, and to help prevent cramping are magnesium chloride and calcium chloride. These ingredients are supplied in amounts calculated to replace the considerable quantities of magnesium and calcium lost in perspiration during exercise. All cations required for or contributing to the prevention or treatment of alkalosis (ammonium ion, potassium ion, magnesium ion, calcium ion) and prevention and treatment of dehydration (sodium ion) were supplied as chloride salts, to maximize the amount of chloride delivered to the subject, as chloride contributes to correcting alkalosis by acting on the kidney. The stimulant ingredients are L-glutamine and green tea extract, which also contribute to immune function. The ingredients used to flavor and color Beverage 3 are elderberry extract and blueberry extract. The flavor/color ingredients were selected to supply intense flavors to mask the taste and mouth feel of the high concentration of electrolytes in this beverage, while also being selected from among natural and organic compounds with immune enhancing properties.

Beverage 3 was prepared by weighing out all the ingredients listed in Table 1, with the exception of the flavor/color ingredients, and dissolving them in 500 ml of water. The solution was mixed gently until all ingredients were fully dissolved. The flavor/color ingredients were then adjusted to achieve the desired flavor and color, and water was added to a final volume of 500 ml.

TABLE 1
Selected formulations of 3 different beverages

| | Dry weight in mg | | |
|---|---|---|---|
| | (for each 500 ml serving) | | |
| | | | Beverage 3 |
| | Beverage 1 | Beverage 2 | (Energizing for |
| Purpose of | (Energizing | (Relaxing | exercise |
| Ingredient | for flights) | for flights) | at high altitude) |
| Treat or | 275 mg | 275 mg | 275 mg |
| prevent | ammonium | ammonium | ammonium |
| alkalosis | chloride | chloride | chloride |
| | 466 mg | 466 mg | 466 mg |
| | potassium | potassium | potassium |
| | chloride | chloride | chloride |
| Treat or | 675 mg | 675 mg | 18,500 mg |
| prevent | D-glucose | D-glucose | sucrose |
| dehydration | 219 mg sodium | 219 mg sodium | 440 mg sodium |
| | chloride | chloride | chloride |
| Multi- | 167 mg | 1,250 mg | 815 mg |
| functional/ | magnesium | magnesium | magnesium |
| treat | chloride | chloride | chloride |
| alkalosis | hexahydrate | hexahydrate | hexahydrate |
| | 140 mg calcium | 140 mg calcium | 685 mg calcium |
| | chloride | chloride | chloride |
| | dihydrate | dihydrate | dihydrate |
| Promote | 250 mg | 25 mg | 250 mg |
| immune | ascorbic acid | ascorbic acid | ascorbic acid |
| function | 4 mg zinc | 8 mg | 20 mg zinc |
| | chloride | zinc chloride | gluconate |
| | 10 μg vitamin | | 10 μg vitamin |
| | D3 | | D3 |
| Immune | 750 mg | 75 mg | 750 mg |
| function and | L-glutamine | 5- | L-glutamine |
| (energy OR | | hydroxytryptophan | |
| relaxation) | | | |
| Flavor/color | green tea | chamomile | elderberry |
| and immune | extract | extract | extract |
| support | sweetened | sweetened ginger | blueberry |
| | pomegranate | extract | extract |
| | extract | | |

### Example 3: Effects of Beverage According to Invention on Arterial Blood Gases (ABG) In a Human Subject

An in vivo experiment was carried out to test one particular embodiment of the composition according to invention (“Test beverage”) and confirm its biochemical effects, particularly on blood gases and blood chemistry, once absorbed orally by a human subject.

Methods: Two servings of Beverage 1 were prepared in 500 ml water each, using the ingredients specified in Table 1. In this example, this embodiment of the invention will be known as the Test beverage.

Arterial blood was collected by a physician from the left radial artery of a human subject in good health. The blood sample (i.e. baseline control sample) was placed on ice and taken for immediate analysis. Shortly after collection of the baseline control blood sample, the subject began consuming a first serving of Test beverage. The first serving of the Test beverage was consumed in approximately 10 minutes. A second, identical serving was consumed over the next 20 minutes, for a total of two servings consumed over approximately 30 minutes. A second sample of arterial blood was collected from the right radial artery one hour after beverage consumption began (i.e. 30 minutes after the end of consumption of the second serving). The blood sample was placed on ice and taken for immediate analysis. In total, 1 hour and 12 minutes elapsed between the two arterial blood draws.

Results: Results of the blood parameters measured in the two arterial blood samples are summarized in Table 2.

As can be seen, the biochemical and blood gas results from the baseline control sample are within the normal ranges for a subject in good health at sea level. After consumption of the two servings of the Test beverage, the bicarbonate levels, base excess, and pH all decreased, demonstrating an acidification of the arterial blood. Furthermore, the arterial partial pressure of carbon dioxide (pCO₂) decreased and the arterial partial pressure of oxygen (pO₂) increased, demonstrating increased ventilation (i.e. a sustained increase in the respiratory rate). Finally, the marked increase in pO₂ after consumption of the Test Beverage was accompanied by an increase in the O₂ saturation. Surprisingly, the pO₂ increased to a value higher than the upper limit of the normal range after consumption of the Test beverage.

TABLE 2
Effects of Test beverage on blood gases and blood chemistry at sea level

| | Before | After | Normal | Observed |
|---|---|---|---|---|
| Parameter | Beverage | Beverage | Range | changes |
| pH | 7.40 | 7.39 | 7.35-7.45 | decrease |
| oxygen saturation | 97 | 99 | | increase |
| (%) | | | | |
| oxygen partial | 89 | 119 | 85-100 | increase |
| pressure (mm | | | | above normal |
| Hg) | | | | |
| CO₂ partial | 39 | 34 | 37-43 | decrease |
| pressure (mm | | | | below normal |
| Hg) | | | | |
| bicarbonate | 25 | 22 | 22-26 | decrease |
| (alkali) (mEq/L) | | | | |
| base excess | −0.5 | −3.6 | | decrease |
| (mEq/L) | | | | |

Discussion: The theoretical model predicted that two servings of a beverage according to the invention, such as the Test beverage used here, would be sufficient to induce measurable decreases in the base excess and blood pH, which in turn would trigger increased respiration, increased dissolved oxygen in the blood (pO₂), and increased oxygen saturation. The results of the arterial blood gas (ABG) analyses performed before and after consumption of the Test beverage confirm these predictions. In fact the invention performed surprisingly better than expected by increasing the amount of dissolved oxygen in the blood even above the normal range. This provides additional evidence for the efficacy of the invention. Concomitantly, the pH remained at a safe level within the normal range (i.e. the pH of arterial blood remained well within the normal range even when two servings of the beverage were consumed quickly at sea level). This provides additional evidence for the safety of the beverage. In addition, the Test beverage lowered the amount of carbon dioxide dissolved in the blood (PCO₂). During air travel, the high ambient level of carbon dioxide due to recycled air and limited cabin space contributes to feelings of anxiety and stress in passengers. Decreasing the PCO₂ may contribute to alleviating these unpleasant sensations. These results clearly confirm the potential of the composition of the invention for modifying blood gases and blood chemistry to counter the undesirable physiological effects of airline travel and high altitude. It is expected that the compositions of the invention will have even more significant effects at high altitude and in flight, where the normal physiological compensatory mechanisms would tend to work with the beverage, thus increasing its effectiveness. Thus, at altitude and during flight, one would predict a greater reduction in blood pH (i.e. starting with an abnormally high value down to a normal value), as well as increased ventilation, increased pO₂, and increased O₂ saturation. Furthermore, normalization of blood pH during flight and at altitude is expected to improve immune function as alkaline pH above the normal range compromises immunity.

### Example 4: Effects of Beverage According to Invention on Oxygen Saturation in a Human Subject During Commercial Air Travel

An in vivo experiment was carried out to test one particular embodiment of the composition according to invention (“Test beverage”) in comparison to a placebo beverage on oxygen saturation of a human subject during commercial airline travel.

Methods: Two servings of Test beverage were prepared in 500 ml water each, using the ingredients for Beverage 1 as specified in Table 1, except that different flavorings were used (elderberry powder instead of green tea extract). In addition, two servings of a placebo beverage were prepared in 500 ml water each, comprising the same vitamins, zinc, glutamine, glucose and flavourings as the Test Beverage, but in which the active ingredients according to the invention (ammonium, potassium, sodium, chloride, magnesium, calcium) were omitted.

During two separate flights of approximately the same duration (2.5 to 3 hours), the same subject consumed the two servings of Test Beverage during the first flight, and the two servings of placebo beverage during the second flight. For each flight, the first serving of beverage was consumed in its entirety beginning in the few minutes before take-off and ending no more than 10 minutes after take-off. The second serving of beverage was consumed slowly beginning one hour after take-off. During each flight, hemoglobin oxygen saturation was monitored at intervals using a CE certified, FDA approved Class II Medical Device fingertip pulse oximeter. At each time point, two measurements were taken; if the measurements were not identical, a third measurement was performed, and the three values were averaged for that time point.

Results: Results of the in-flight pulse oximetry during the two flights (Test beverage flight and placebo flight) are illustrated in FIG. 2.

For the flight during which the placebo beverage was consumed, oxygen saturation decreased from a normal sea level value of 99% down to 92-93%. This is as expected according to previous reports in the scientific literature. In contrast, during the flight in which the Test beverage was consumed, oxygen saturation remained high: all oxygen saturation measurements were 97% or higher.

Discussion: During commercial airline travel, passenger hemoglobin oxygen saturation routinely decreases from normal sea-level values of approximately 97-99% down to approximately 92-94%. However, a beverage prepared according to one embodiment of the invention, and consumed in sufficient amounts during air travel, prevented this drop of hemoglobin oxygen saturation, and kept the oxygen saturation at 97% or higher. Furthermore, the data show that during the placebo flight, the subject's oxygen saturation cycled up and down between 96% and 92.5%. This is expected according to standard models of respiratory physiology, and represents an autonomic increase in respiratory rate as blood CO₂ levels increase (and oxygen levels decrease), followed by slowing of the respiratory rate as CO₂ is “blown off”. This cycling in respiratory rates, and concomitant cyclical changes in oxygen saturation and downstream disturbances of blood pH and electrolytes, contribute to discomfort and difficulty sleeping at high altitude and during air travel. The Test beverage according to invention abolished the overall drop in oxygen saturation, as well as the cycling up and down of oxygen saturation, and resulted in a stable oxygen saturation sustained at 97% or above. Maintaining stable, increased levels of oxygen saturation throughout the flight can help prevent discomfort and fatigue felt by passengers during air travel, and is likely to improve sleep quality during overnight flights.

### Example 5. Comparison of the Effects on Oxygen Saturation of Beverages According to Invention Versus Beverages Served by Airlines: Data from Multiple Commercial Flights

Three subjects measured their oxygen saturation during a total of 8 commercial airline flights during which they consumed either complimentary beverages served in-flight (water, fruit juice, and/or coffee), or Test beverages according to embodiments of the invention. Oxygen saturation data from control flights (complimentary in-flight beverages only) were compared with those from Test beverage flights.

Methods: Each subject participated in one control flight during which they drank beverages of their choice supplied by the airline (control flights), and one or two flights during which they drank two doses per flight of Test beverages. Test beverages were prepared according to Table 1, Beverage 1, except that each beverage was flavoured and sweetened with several alternative flavour/sweetener preparations according to the subjects' preference (for example, pomegranate, elderberry, citrus, etc). Each subject measured his or her oxygen saturation at least 3 times, one hour or more after take-off and before the plane began final descent, and the mean value from the 3 measurements was recorded. All flights were short-haul (duration, 2 to 3 hours flight time), and data were collected from a total of 8 flights. Data were pooled for Control Beverage versus Test Beverage flights and analysed for significance with a two-tailed Student's t-test.

Results: Results are shown in FIG. 3. When drinking control beverages (water, juice, and/or coffee), all subjects experienced a drop in oxygen saturation to 93% or below. In all subjects, oxygen saturation was markedly improved on flights during which the Test Beverage was consumed. The improvement in oxygen saturation produced by the invention was highly statistically significant (p<0.0001, see FIG. 3).

Discussion: Taken together, examples 4 and 5 demonstrate that embodiments of the invention can largely prevent the drop in hemoglobin oxygen saturation caused by commercial airline travel, thereby protecting passengers from the physiologically undesirable effects of abnormally low oxygen saturation and the accompanying decreased oxygen delivery to the tissues, which can lead to symptoms such as headache and fatigue.

### Example 6. Kinetics of the Effects on Oxygen Saturation of a Beverage According to Invention During a Long-Haul Flight

The duration of effectiveness for maintaining high oxygen saturation (about 97% or above) of one embodiment according to invention was determined by using pulse oximetry to monitor oxygen saturation of a subject following consumption at intervals, in divided doses, of several servings of a beverage prepared according to the invention, during a long-haul commercial flight of just over 6 hours.

Methods: To determine the length of time during which one portion of beverage prepared according to Table 1, Beverage 1, would maintain a high oxygen saturation (about 97% and above), a subject consumed Test beverage one-half portion at a time (equivalent to 250 ml of beverage, see Table 1, Beverage 1), and oxygen saturation was monitored by pulse oximetry. During the six hour 20 minute flight, 2.5 servings of Test beverage were consumed (0.5 portion immediately before takeoff, and the remaining 2 portions in 4 half-portion doses at intervals during the flight), and twenty-seven oxygen saturation measurements were performed.

Results: A chart of the data is shown in FIG. 4.

The pulse oximetry data indicate that after each half-dose of Test beverage was consumed, a rapid (within 5 to 10 minutes) increase in oxygen saturation occurred, and was sustained for about 50 to 80 minutes. Thus, one half-portion of Test beverage conferred protection from low oxygen saturation (below about 97%) in-flight for approximately one hour in this subject, and a full portion offered protection for two to three hours. The results suggest that there may be a cumulative effect with the second dose remaining effective for longer, or preserving a higher level of oxygen saturation, than the first dose.

Discussion: These empirical data agree well with the theoretical kinetics predicted by the mathematical model described in Example 1. These results support the effectiveness of one dose prepared as described in Table 1, beverage 1, for a short-haul flight of less than 3 hours, and suggest that for maximum effectiveness during long-haul flights, an additional serving should be consumed for each additional 2 to 3 hours of flying time.

### Example 7. Effects of a Beverage According to Invention on Subjective Ratings of Airline Passenger Comfort

During round-trip, short-haul flights, subjects consumed Test beverages (prepared according to the invention) during one flight segment and control beverages of their choice during the other (return) segment. The subjects rated six subjective comfort-related parameters at the beginning and end of each flight (control and Test flights). Results with and without the Test beverage were compared to determine the subjective effects of the Test Beverage on passenger comfort.

Methods: Several embodiments of the invention were prepared according to Table 1, Beverage 1 and Beverage 2, using the ingredients listed with the exception of different flavourings and/or sweeteners depending on the subjects' preferences (all of these beverages were prepared according to the invention and are referred to as Test beverages). Subjects traveled on round-trip non-stop flights of 2 to 3 hours duration. Subjects were asked to consume one 500 ml serving of Test beverage during one flight segment, and to drink beverages of their choice on the other (return) flight segment. At the beginning and end of each flight they were asked to grade six parameters (general wellbeing, absence of headache, hydration, muscle comfort, energy, and alertness) on a visual analogue scale.

For each test subject, a score for each parameter was calculated by subtracting the starting value from the end value, to determine the change in that parameter during each flight. A negative value indicates a reduction in that parameter during the course of the flight. For example, if the subject began the flight with a “general wellbeing” value of 5 and ended with a value of 3, their sense of wellbeing would have decreased by 2 arbitrary units during the flight and their score for general wellbeing on that flight would be −2 (negative 2). The change in each comfort parameter for each passenger could then be compared for flights with or without the Test beverage (FIG. 5). This approach can detect prevention or partial alleviation of specific unpleasant symptoms in travelers who habitually experience discomfort when they fly, as well as a net increase in comfort in passengers who normally have few subjective symptoms during air travel (FIG. 5).

A global comfort score was then calculated by adding together the 6 subscores (for general wellbeing, absence of headache, hydration, muscle comfort, energy, and alertness), to generate an overall score for the Test beverage flight and an overall score for the control flight for each subject, as shown in FIG. 6 (a). The global scores for the control flights and the test flights were then averaged, and statistical significance was determined with a two-tailed paired Student's t-test, shown in FIG. 6 (b).

Results: The complete dataset for one subject, illustrating the change in all 6 comfort parameters during the control flight (without Test beverage, hashed bars) and the test flight (with Test beverage according to invention, solid black bars) is shown in FIG. 5. The Test beverage for this subject was prepared according to Beverage 2, Table 1 (relaxing beverage for flights). The control and test flights were the two segments of a round trip flight. During the course of the control flight, this subject experienced decreased wellbeing, worsening headache, dehydration, muscle discomfort, and diminishing alertness. In contrast, during the Test beverage flight, the same subject reported that wellbeing increased during the flight, with decreasing headache, better hydration, improved relaxation (muscle comfort) and more energy at the end of the test flight than at its beginning. FIG. 6 shows the global comfort scores (an aggregate of the 6 parameters illustrated individually in FIG. 5) for 3 subjects. Subjects 1 and 3 consumed Test beverages prepared according to Beverage 1, Table 1 (energizing beverage for flights); and subject 2 received a Test beverage prepared according to Beverage 2, Table 1 (relaxing beverage for flights).

Subject 1 habitually experiences substantial discomfort during air travel. During the control flight, this subject reported worsening headache, dehydration, and loss of alertness (FIG. 6 (a), only global score is shown). In contrast, with the Test beverage, this subject felt essentially the same at the end of the flight as at the beginning; the test beverage largely prevented the unpleasant symptoms usually experienced by this subject. Subjects 2 and 3 also experienced discomfort during the control flights, although to a lesser extent than subject 1. In these two subjects, the Test beverage not only prevented the discomfort felt during control flights, but in addition, left them feeling better at the end of the test flight than at its beginning (see FIG. 6 (a), subjects 2 and 3).

FIG. 6 (b) shows the mean and standard error of the global comfort score for all subjects. There is a wide variation in the subjective experience of discomfort during air travel. However, all subjects reported improvement with the Test beverage compared to control flights, and of note, the degree of improvement (change in comfort Δ during the flight) was surprisingly consistent between subjects (ie. subject 1 improved from a control value of −19 up to a test flight value of −1, Δ=18; subject 2 went from −10 to +6 with the Test beverage, Δ=16; and subject 3, from −13 to +4, Δ=17). Furthermore, the improvement in the mean global comfort scores with Test beverage compared with control was statistically significantly (p=0.0015, FIG. 6 (b)).

Discussion: Several embodiments of the invention mitigated, prevented or even reversed undesirable subjective symptoms of air travel as self-reported by passengers, including improvements in general wellbeing; alleviation of headache, dehydration, and muscle discomfort; and enhanced energy and alertness. Although individual subjective assessment of discomfort and wellbeing varied substantially, all subjects reported improvement with the Test beverage according to invention, and in general, subjects who normally experienced many undesirable effects during flight reported that they felt immediately better after consuming the Test beverage and found their symptoms greatly alleviated throughout the flight, while subjects with fewer symptoms to begin with often reported actually feeling better at the end of the flight than at its beginning when they consumed the Test beverage.

Together, the results presented in Examples 3 through 7 support the effects of the compositions of the invention in alleviating the undesirable physiological effects of low partial pressure of oxygen existing in flight and at altitudes above sea level. These results demonstrate that ingesting the composition of the invention have the following effects: 1, increases the oxygen dissolved in arterial blood (PaO₂) and reduces the carbon dioxide (PaCO₂); 2, regulates the acid-base balance (pH) of the blood by decreasing the base excess and thus counteracts the excess alkalinisation that occurs at low ambient air pressure; 3 increases the hemoglobin oxygen saturation during flight; 4, increases passenger's subjective assessments of comfort and reduces subjective unpleasant symptoms during flight, including increased general wellbeing, reduced headache, improved hydration, improved muscle comfort (relaxation), improved alertness and energy. In addition, by normalizing biochemical parameters including blood oxygen levels, electrolytes, and pH, and by fostering a sense of improved comfort and wellbeing, the composition of the invention is likely to protect passengers' health through multiple physiological and psychological mechanisms.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

Consumable compositions and uses thereof for alleviating undesirable physiological effects systems 100 may be manufactured and provided for sale in a wide variety of sizes and shapes for a wide assortment of applications.

Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

It should be noted that the steps described in the method of use can be carried out in many different orders according to user preference. The use of “step of” should not be interpreted as “step for”, in the claims herein and is not intended to invoke the provisions of 35 U.S.C. §112, §6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

1) A consumable composition comprising ammonium;
a) wherein said consumable composition comprising said ammonium is formulated for human consumption; and
b) wherein said consumable composition is useful for alleviating undesirable physiological effects caused by exposure to low air pressure which occurs during air travel and alternatively at altitudes above 5,000 feet.

2) The consumable composition of claim 1 useful for alleviating said undesirable physiological effects comprising alkalosis.

3) The consumable composition of claim 1 useful for alleviating said undesirable physiological effects comprising hypoxia.

4) The consumable composition of claim 1 further comprising chloride and potassium.

5) The consumable composition of claim 4, wherein a combination of said ammonium, said chloride, and said potassium is useful for alleviating alkalosis and alternatively hypoxia.

6) A consumable composition comprising:
a) at least about 0.006% w/v ammonium;
b) at least about 0.04% w/v chloride; and
c) at least about 0.15% w/v potassium;
d) wherein said at least about 0.006% w/v ammonium, said at least about 0.04% w/v chloride, and said at least about 0.15% w/v potassium comprises in chemical combination said consumable composition;
e) wherein said consumable composition is formulated for human consumption; and
f) wherein said consumable composition is useful for alleviating undesirable physiological effects.

7) The consumable composition of claim 6 further comprising sodium.

8) The consumable composition of claim 6 further comprising at least one sweetener and alternatively at least one sugar.

9) The consumable composition of claim 6 further comprising at least one flavoring agent useful for adding flavor to said consumable composition.

10) The consumable composition of claim 6 further comprising at least one coloring agent useful for adding color to said consumable composition.

11) The consumable composition of claim 6 further comprising at least one preservative useful for preserving said consumable composition over a period of time.

12) A container comprising consumable composition of claim 6 and further comprising:
a) a container body; and
b) an inner volume;
c) wherein said container body and said inner volume structurally comprise said container for retaining said consumable composition;
d) wherein said consumable composition comprises ammonium chloride and potassium chloride;
e) wherein said ammonium chloride and said potassium chloride function in chemical combination for alleviating undesirable physiological effects; and
f) wherein said container body proximate to said ammonium chloride and said potassium chloride is useful for preserving an efficacy of said consumable composition.

13) The container for retaining said consumable composition of claim 12 wherein said consumable composition is useful for human consumption.

14) The container for retaining said consumable composition of claim 13 wherein said container body further comprises sodium useful for preserving said efficacy of said consumable composition.

15) The container for retaining said consumable composition of claim 13 wherein said container body further comprises magnesium useful for preserving said efficacy of said consumable composition.

16) The container for retaining said consumable composition of claim 13 wherein said container body further comprises calcium useful for preserving said efficacy of said consumable composition.

17) The container for retaining said consumable composition of claim 13 wherein said container body further comprises zinc useful for preserving said efficacy of said consumable composition.

18) The container for retaining said consumable composition of claim 13 wherein said container body comprises a package and alternatively a pouch useful for retaining said consumable composition comprising a non-aqueous solution.

19) The container for retaining said consumable composition of claim 13 wherein said container body comprises a can and alternatively a bottle useful for retaining said consumable composition comprising an aqueous solution.

20) The container for retaining said consumable composition of claim 13 comprising;
a) at least two said container bodies;
b) each container body(s) comprising said consumable composition; and
c) a set of user instructions.
